(19) United States
(12) Patent Application Publication
(10) Pub. No.: US 2011/0172244 A1
(43) Pub. Date: Jul. 14, 2011

(54) COMPOUNDS AND COMPOSITIONS AS MODULATORS OF GPR119 ACTIVITY

(75) Inventors: Phillip B. Alper, San Diego, CA (US); Robert Epple, San Diego, CA (US); Pierre-Yves Michellys, San Diego, CA (US); Daniel Mutnick, San Diego, CA (US); Victor Nikulin, Carlsbad, CA (US)

(73) Assignee: IRM LLC, Hamilton (BM)

(21) Appl. No.: 12/918,804

(22) PCT Filed: Feb. 20, 2009
(86) PCT No.: PCT/US09/34789

§ 371 Date: Feb. 23, 2011

Related U.S. Application Data
(60) Provisional application No. 61/030,897, filed on Feb. 22, 2008.

(51) Int. Cl. A61K 31/496; C07D 401/06; A61K 31/454; C07D 405/06; A61K 31/4525; C07D 403/06; C07D 405/14; C07D 401/14; C07D 209/42; A61K 31/404; C12N 5/071; A61P 3/04; A61P 3/10; A61P 3/06; A61P 9/00; A61P 9/10; A61P 3/08; A61P 19/02; A61P 19/10; A61P 9/12; A61P 9/04; A61P 27/02; A61P 13/12; A61P 3/00; A61P 7/02; A61P 19/04; A61P 17/00; A61P 15/00

(52) U.S. Cl. 514/253.09; 546/201; 514/323; 546/196; 514/320; 544/373; 514/254.09; 544/364; 548/484; 514/418; 544/376; 514/254.11; 435/375

(57) ABSTRACT

The invention provides compounds, of Formula I: pharmaceutical compositions comprising such compounds and methods of using such compounds to treat or prevent diseases or disorders associated with the activity of GPR119.

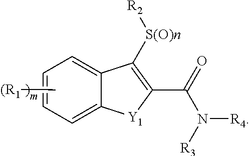

### CROSS-REFERENCE TO RELATED APPLICATIONS

This application claims the benefit of priority to U.S. Provisional Patent Application No. 61/030,897, filed 22 Feb. 2008. The full disclosure of this application is incorporated herein by reference in its entirety and for all purposes.

### BACKGROUND OF THE INVENTION

1. Field of the Invention

The invention provides compounds, pharmaceutical compositions comprising such compounds and methods of using such compounds to treat or prevent diseases or disorders associated with the activity of GPR119.

2. Background

GPR119 is a G-protein coupled receptor (GPCR) that is mainly expressed in the pancreas, small intestine, colon and adipose tissue. The expression profile of the human GPR119 receptor indicates its potential utility as a target for the treatment of obesity and diabetes. The novel compounds of this invention modulate the activity of GPR119 and are, therefore, expected to be useful in the treatment of GPR119-associated diseases or disorders such as, but not limited to, diabetes, obesity and associated metabolic disorders.

### SUMMARY OF THE INVENTION

In one aspect, the present invention provides a compound of Formula I:

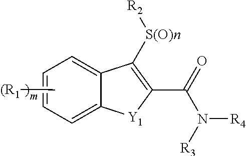

in which:

m is selected from 0, 1, 2, 3 and 4;

n is selected from 0, 1 and 2;

R₁ is selected from halo, halo-substituted-C₁₋₆alkyl, C₁₋₆alkoxy, halo-substituted-C₁₋₆alkoxy and C₁₋₆alkyl;

R₂ is selected from C₆₋₁₀aryl-C₀₋₄alkyl, C₅₋₁₀heteroaryl-C₀₋₄alkyl, C₃₋₁₂cycloalkyl-C₀₋₄alkyl, C₃₋₈heterocycloalkyl-C₀₋₄alkyl and C₁₋₆alkyl; wherein any aryl, heteroaryl, cycloalkyl, heterocycloalkyl or alkyl of R₂ can be optionally substituted with 1 to 3 radicals independently selected from halo, halo-substituted-C₁₋₆alkyl, C₁₋₆alkoxy, halo-substituted-C₁₋₆alkoxy, C₁₋₆alkyl and C₆₋₁₀aryl;

R₃ is selected from hydrogen and C₁₋₆alkyl;

R₄ is selected from —X₁R₅ and —X₁OR₅; wherein X₁ is selected from a bond, —C(O)—, —NR₆— and C₁₋₆alkylene; R₅ is selected from C₆₋₁₀aryl, C₁₋₁₀heteroaryl, C₃₋₈heterocycloalkyl and C₃₋₁₂cycloalkyl; R₆ is selected from hydrogen and C₁₋₆alkyl;

or R₃ and R₄ together with the nitrogen atom to which R₃ and R₄ are attached form C₃₋₈heterocycloalkyl; wherein said aryl or cycloalkyl of R₄ or said heterocycloalkyl of the combination of R₃ and R₄ can be optionally substituted with 1 to 3 radicals independently selected from —X₂R₇, —X₂C(O)R₇, —X₂S(O)₀₋₂R₇—X₂NR₈X₃R₇ and —X₂OR₇; wherein X₂ and X₃ are independently selected from a bond and C₁₋₄alkylene; R₇ is selected from C₆₋₁₀aryl, C₃₋₁₂cycloalkyl, C₁₋₁₀heteroaryl and C₃₋₈heterocycloalkyl; wherein said aryl, heteroaryl, cycloalkyl and heterocycloalkyl of R₇ is optionally substituted with 1 to 3 radicals independently selected from halo, hydroxy, nitro, cyano, halo-substituted-C₁₋₆alkyl, C₁₋₆alkoxy, halo-substituted-C₁₋₆alkoxy, C₁₋₆alkyl, C₆₋₁₀aryl and —C(O)R₉; wherein R₈ is selected from hydrogen and C₁₋₆alkyl; R₉ is selected from hydrogen and C₁₋₆alkyl;

Y₁ is selected from O and NR₁₀; wherein R₁₀ is selected from hydrogen and C₁₋₆alkyl.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which modulation of GPR119 activity can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which GPR119 activity contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be straight-chained, branched, cyclic or spiro. C₁₋₆alkoxy includes methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl can be phenyl or naphthyl, preferably phenyl. “Arylene” means a divalent radical derived from an aryl group. “Heteroaryl” is as defined for aryl where one or more of the ring members are a heteroatom. For example, C₁₋₁₀heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, 1H-pyridin-2-onyl, 6-oxo-1,6-dihydro-pyridin-3-yl, etc. “C₆₋₁₀arylC₀₋₄alkyl” means an aryl as described above connected via a alkylene grouping. For example, C₆₋₁₀arylC₀₋₄alkyl includes phenethyl, benzyl, etc. Heteroaryl also includes the N-oxide derivatives, for example, pyridine N-oxide derivatives with the following structure:

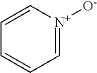

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc. “Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, 2-oxo-pyrrolidin-1-yl, 2-oxo-piperidin-1-yl, etc.

GPR119 means G protein-coupled receptor 119 (GenBank® Accession No. AAP72125) is also referred to in the literature as RUP3 and GPR116. The term GPR119 as used herein includes the human sequences found in GeneBank accession number AY288416, naturally-occurring allelic variants, mammalian orthologs, and recombinant mutants thereof.

“Halogen” (or halo) preferably represents chloro or fluoro, but can also be bromo or iodo.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of diseases in which modulation of GPR119 activity can prevent, inhibit or ameliorate the pathology and/or symptomology of the diseases, which method comprises administering to the animal a therapeutically effective amount of a compound of Formula I.

In one embodiment, with reference to compounds of Formula I, are compounds in which:

m is selected from 0, 1 and 2;

n is selected from 0, 1 and 2;

R₁ is selected from halo, halo-substituted-C₁₋₆alkyl, C₁₋₆alkoxy, halo-substituted-C₁₋₆alkoxy and C₁₋₆alkyl;

R₂ is selected from C₆₋₁₀aryl-C₀₋₄alkyl, C₃₋₁₂cycloalkyl-C₀₋₄alkyl, and C₁₋₆alkyl; wherein any aryl, cycloalkyl or alkyl of R₂ can be optionally substituted with 1 to 3 radicals independently selected from halo, halo-substituted-C₁₋₆alkyl, C₁₋₆alkoxy, halo-substituted-C₁₋₆alkoxy and C₁₋₆alkyl;

R₃ is selected from hydrogen and C₁₋₆alkyl;

R₄ is selected from —X₁R₅ and —X₁OR₅; wherein X₁ is selected from a bond, —C(O)—, —NR₆— and C₁₋₆alkylene; R₅ is selected from C₆₋₁₀aryl, C₁₋₁₀heteroaryl and C₃₋₁₂cycloalkyl; R₆ is selected from hydrogen and C₁₋₆alkyl;

or R₃ and R₄ together with the nitrogen atom to which R₃ and R₄ are attached form C₃₋₈heterocycloalkyl; wherein said aryl or cycloalkyl of R₄ or said heterocycloalkyl of the combination of R₃ and R₄ can be optionally substituted with 1 to 3 radicals independently selected from —X₂R₇, —X₂C(O)R₇, —X₂S(O)₀₋₂R₇—X₂NR₈X₃R₇ and —X₂OR₇; wherein X₂ and X₃ are independently selected from a bond and C₁₋₄alkylene; R₇ is selected from C₆₋₁₀aryl, C₃₋₁₂cycloalkyl, C₁₋₁₀heteroaryl and C₃₋₈heterocycloalkyl; wherein said aryl, heteroaryl, cycloalkyl and heterocycloalkyl of R₇ is optionally substituted with 1 to 3 radicals independently selected from halo, hydroxy, nitro, cyano, halo-substituted-C₁₋₆alkyl, C₁₋₆alkoxy, halo-substituted-C₁₋₆alkoxy, C₁₋₆alkyl, C₆₋₁₀aryl and —C(O)R₉; wherein R₈ is selected from hydrogen and C₁₋₆alkyl; R₉ is selected from hydrogen and C₁₋₆alkyl; and

Y₁ is selected from O and NR₁₀; wherein R₁₀ is selected from hydrogen and C₁₋₆alkyl.

In another embodiment, R₁ is selected fluoro, chloro, bromo, methyl, trifluoromethyl and methoxy; and R₂ is selected from phenyl, benzyl, cyclohexyl, phenethyl and isopentyl; wherein said phenyl, benzyl, cyclohexyl and phenethyl is optionally substituted with a halo radical.

In another embodiment, R₃ is selected from hydrogen and methyl; and R₄ is selected from phenoxy-ethyl, benzyl, phenethyl, phenyl-butyl, biphenyl and cyclohexyl-methyl; or R₃ and R₄ together with the nitrogen atom to which R₃ and R₄ are attached form piperidinyl or piperazinyl; wherein said piperidinyl or piperazinyl are optionally substituted with a group selected from benzyl, phenoxy, phenyl-thio, phenethyl, cyclohexyl-methyl, benzo[d][1,3]dioxolyl-methyl, phenyl-carbonyl, benzyl-(methyl)-amino and pyridinyl-methyl; wherein said benzyl, phenoxy, phenyl-thio, phenethyl, cyclohexyl-methyl, benzo[d][1,3]dioxolyl-methyl, phenyl-carbonyl, benzyl-(methyl)-amino or pyridinyl-methyl substituents of the combination of R₃ and R₄ are further optionally substituted by 1 to 3 radicals independently selected from chloro, fluoro, bromo, methyl, trifluoromethyl, t-butyl, methoxy and formyl.

In another embodiment, Y₁ is selected from O and NR₁₀; wherein R₁₀ is selected from hydrogen and methyl.

In another embodiment are compounds selected from: (4-benzylpiperidin-1-yl)(3-(4-chlorophenylsulfonyl)-5-fluoro-1H-indol-2-yl)methanone; (3-(4-Chlorophenylsulfonyl)benzofuran-2-yl)(4-(4-(trifluoromethyl)phenoxy)piperidin-1-yl)methanone; (4-(2-chloro-6-fluorobenzyl)piperazin-1-yl)(3-(4-chlorophenylsulfonyl)-1H-indol-2-yl)methanone; (3-(4-chlorophenylsulfonyl)-1H-indol-2-yl)(4-(p-tolyloxy)piperidin-1-yl)methanone; (3-(4-chlorophenylsulfonyl)-1H-indol-2-yl)(4-(4-(trifluoromethyl)phenoxy)piperidin-1-yl)methanone; (4-(4-chlorobenzyl)piperazin-1-yl)(3-(4-chlorophenylsulfonyl)-1H-indol-2-yl)methanone; (3-(4-chlorophenylsulfonyl)-1H-indol-2-yl)(4-(p-tolylthio)piperidin-1-yl)methanone; (3-(4-chlorophenylsulfonyl)-1H-indol-2-yl)(4-phenethylpiperidin-1-yl)methanone; (4-(4-tert-butylbenzyl)piperazin-1-yl)(3-(4-chlorophenylsulfonyl)-1H-indol-2-yl)methanone; (3-(4-chlorophenylsulfonyl)-1H-indol-2-yl)(4-(4-methoxybenzyl)piperazin-1-yl)methanone; (4-benzylpiperazin-1-yl)(3-(4-chlorophenylsulfonyl)-1H-indol-2-yl)methanone; (3-(4-chlorophenylsulfonyl)-1H-indol-2-yl)(4-(cyclohexylmethyl)piperazin-1-yl)methanone; (4-(benzo[d][1,3]dioxol-5-ylmethyl)piperazin-1-yl)(3-(4-chlorophenylsulfonyl)-1H-indol-2-yl)methanone; (4-benzylpiperidin-1-yl)(5-chloro-3-(4-chlorophenylsulfonyl)-1H-indol-2-yl)methanone; (4-benzoylpiperidin-1-yl)(3-(4-chlorophenylsulfonyl)-1H-indol-2-yl)methanone; (4-benzylpiperidin-1-yl)(3-(4-chlorophenylsulfonyl)-5,7-difluoro-1H-indol-2-yl)methanone; 4-(1-(3-(4-chlorophenylsulfonyl)-1H-indole-2-carbonyl)piperidin-4-yloxy)benzaldehyde; (3-(4-chlorophenylsulfonyl)-1H-indol-2-yl)(4-phenethylpiperazin-1-yl)methanone; (4-benzylpiperidin-1-yl)(3-(4-chlorophenylsulfonyl)-7-(trifluoromethyl)-1H-indol-2-yl)methanone; (4-(benzyl(methyl)amino)piperidin-1-yl)(3-(4-chlorophenylsulfonyl)-1H-indol-2-yl)methanone; 3-(4-chlorophenylsulfonyl)-N-methyl-N-(2-phenoxyethyl)-1H-indole-2-carboxamide; (4-benzylpiperidin-1-yl)(5-bromo-3-(4-chlorophenylsulfonyl)-1H-indol-2-yl)methanone; (3-(4-chlorophenylsulfonyl)-1H-indol-2-yl)(4-(pyridin-4-ylmethyl)piperazin-1-yl)methanone; 3-(4-chlorophenylsulfonyl)-N-(4-phenylbutyl)-1H-indole-2-carboxamide; (3-(4-chlorophenylsulfonyl)-1H-indol-2-yl)(piperidin-1-yl)methanone; 3-(4-chlorophenylsulfonyl)-N-methyl-N-phenethyl-1H-indole-2-carboxamide; N-benzyl-3-(4-chlorophenylsulfonyl)-N-methyl-1H-indole-2-carboxamide; N-(biphenyl-4-yl)-3-(4-chlorophenylsulfonyl)-1H-indole-2-carboxamide; 3-(4-chlorophenylsulfonyl)-N-(cyclohexylmethyl)-1H-indole-2-carboxamide; (4-benzylpiperazin-1-yl)(3-(4-chlorophenylsulfonyl)benzofuran-2-yl)methanone; (4-benzylpiperidin-1-yl)(3-(4-chlorophenylsulfonyl)benzofuran-2-yl)methanone; (4-benzylpiperidin-1-yl)(3-(4-chlorophenylsulfonyl)-1H-indol-2-yl)methanone; (4-benzylpiperidin-1-yl)(3-(4-chlorophenylsulfonyl)-7-fluoro-1H-indol-2-yl)methanone; (4-benzylpiperidin-1-yl)(3-benzylsulfonyl-1H-indol-2-yl)methanone; (4-benzylpiperidin-1-yl)(3-(cyclohexylthio)-1H-indol-2-yl)methanone; (4-benzylpiperidin-1-yl)(3-benzylsulfonyl-1H-indol-2-yl)methanone; (4-Benzylpiperidin-1-yl)(3-(4-chlorophenylsulfonyl)-1-methyl-1H-indol-2-yl)methanone; (4-benzylpiperidin-1-yl)(7-chloro-3-(4-chlorophenylsulfonyl)-1H-indol-2-yl)methanone; (4-benzylpiperidin-1-yl)(5,7-dichloro-3-(4-chlorophenylsulfonyl)-1H-indol-2-yl)methanone; (4-benzylpiperidin-1-yl)(3-(4-chlorophenylsulfonyl)-7-methoxy-1H-indol-2-yl)methanone; (4-benzylpiperidin-1-yl)(3-(4-chlorophenylsulfonyl)-5-methyl-1H-indol-2-yl)methanone; (4-benzylpiperidin-1-yl)(3-(phenethylsulfonyl)-1H-indol-2-yl)methanone; (4-benzylpiperidin-1-yl)(3-(isopentylsulfonyl)-1H-indol-2-yl)methanone; (4-Benzylpiperidin-1-yl)(3-(phenylthio)-1H-indol-2-yl)methanone; (4-benzylpiperidin-1-yl)(3-(4-chlorophenylthio)-1H-indol-2-yl)methanone; and (4-benzylpiperazin-1-yl)(3-(4-chlorophenylthio)-1H-indol-2-yl)methanone.

Further compounds of the invention are detailed in the Examples and Table I, infra.

The present invention also includes all suitable isotopic variations of the compounds of the invention, or pharmaceutically acceptable salts thereof. An isotopic variation of a compound of the invention or a pharmaceutically acceptable salt thereof is defined as one in which at least one atom is replaced by an atom having the same atomic number but an atomic mass different from the atomic mass usually found in nature. Examples of isotopes that may be incorporated into the compounds of the invention and pharmaceutically acceptable salts thereof include but are not limited to isotopes of hydrogen, carbon, nitrogen and oxygen such as ²H, ³H, ¹¹C, ¹³C, ¹⁴C, ¹⁵N, ¹⁷O, ¹⁸O, ³⁵S, ¹⁸F, ³⁶Cl and ¹²³I. Certain isotopic variations of the compounds of the invention and pharmaceutically acceptable salts thereof, for example, those in which a radioactive isotope such as ³H or ¹⁴C is incorporated, are useful in drug and/or substrate tissue distribution studies. In particular examples, ³H and ¹⁴C isotopes may be used for their ease of preparation and detectability. In other examples, substitution with isotopes such as ²H may afford certain therapeutic advantages resulting from greater metabolic stability, such as increased in vivo half-life or reduced dosage requirements. Isotopic variations of the compounds of the invention or pharmaceutically acceptable salts thereof can generally be prepared by conventional procedures using appropriate isotopic variations of suitable reagents.

### Pharmacology and Utility

Compounds of the invention modulate the activity of GPR119 and, as such, are useful for treating diseases or disorders in which the activity of GPR119 contributes to the pathology and/or symptomology of the disease. This invention further provides compounds of this invention for use in the preparation of medicaments for the treatment of diseases or disorders in which GPR119 activity contributes to the pathology and/or symptomology of the disease.

The resultant pathologies of Type II diabetes are impaired insulin signaling at its target tissues and failure of the insulin-producing cells of the pancreas to secrete an appropriate degree of insulin in response to a hyperglycemic signal. Current therapies to treat the latter include inhibitors of the β-cell ATP-sensitive potassium channel to trigger the release of endogenous insulin stores, or administration of exogenous insulin. Neither of these achieves accurate normalization of blood glucose levels and both carry the risk of inducing hypoglycemia. For these reasons, there has been intense interest in the development of pharmaceuticals that function in a glucose-dependent action, i.e. potentiators of glucose signaling. Physiological signaling systems which function in this manner are well-characterized and include the gut peptides GLP-1, GIP and PACAP. These hormones act via their cognate G-protein coupled receptor to stimulate the production of cAMP in pancreatic β-cells. The increased cAMP does not appear to result in stimulation of insulin release during the fasting or pre-prandial state. However, a series of biochemical targets of cAMP signaling, including the ATP-sensitive potassium channel, voltage-sensitive potassium channels and the exocytotic machinery, are modified in such a way that the insulin secretory response to a postprandial glucose stimulus is markedly enhanced. Accordingly, agonists of novel, similarly functioning, β-cell GPCRs, including GPR119, would also stimulate the release of endogenous insulin and consequently promote normoglycemia in Type II diabetes. It is also established that increased cAMP, for example as a result of GLP-1 stimulation, promotes β-cell proliferation, inhibits β-cell death and thus improves islet mass. This positive effect on β-cell mass is expected to be beneficial in both Type II diabetes, where insufficient insulin is produced, and Type I diabetes, where β-cells are destroyed by an inappropriate autoimmune response.

Some β-cell GPCRs, including GPR119, are also present in the hypothalamus where they modulate hunger, satiety, decrease food intake, controlling or decreasing weight and energy expenditure. Hence, given their function within the hypothalamic circuitry, agonists or inverse agonists of these receptors mitigate hunger, promote satiety and therefore modulate weight.

It is also well-established that metabolic diseases exert a negative influence on other physiological systems. Thus, there is often the codevelopment of multiple disease states (e.g. type I diabetes, type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which clearly occur secondary to diabetes (e.g. kidney disease, peripheral neuropathy). Thus, it is expected that effective treatment of the diabetic condition will in turn be of benefit to such interconnected disease states.

In an embodiment of the invention is a method for treatment of a metabolic disease and/or a metabolic-related disorder in an individual comprising administering to the individual in need of such treatment a therapeutically effective amount of a compound of the invention or a pharmaceutical composition thereof. The metabolic diseases and metabolic-related disorders are selected from, but not limited to, hyperlipidemia, type 1 diabetes, type 2 diabetes mellitus, idiopathic type 1 diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance.

In an embodiment of the invention are therapeutic benefits of GPR119 activity modulators derived from increasing levels of GIP and PPY. For example, neuroprotection, learning and memory, seizures and peripheral neuropathy.

GLP-1 and GLP-1 receptor agonists have been shown to be effective for treatment of neurodegenerative diseases and other neurological disorders. GLP-1 and exendin-4 have been shown to stimulate neurite outgrowth and enhance cell survival after growth factor withdrawal in PC12 cells. In a rodent model of neurodegeneration, GLP-1 and exendin-4 restore cholinergic marker activity in the basal forebrain. Central infusion of GLP-1 and exendin-4 also reduce the levels of amyloid-β peptide in mice and decrease amyloid precursor protein amount in cultured PC12 cells. GLP-1 receptor agonists have been shown to enhance learning in rats and the GLP-1 receptor knockout mice show deficiencies in learning behavior. The knockout mice also exhibit increased susceptibility to kainate-induced seizures which can be prevented by administration of GLP-1 receptor agonists. GLP-1 and exendin-4 has also been shown to be effective in treating pyridoxine-induced peripheral nerve degeneration, an experimental model of peripheral sensory neuropathy.

Glucose-dependent insulinotropic polypeptide (GIP) has also been shown to have effects on proliferation of hippocampal progenitor cells and in enhancing sensorimotor coordination and memory recognition.

In an embodiment of the invention are therapeutic benefits of GPR119 activity modulators. For example, GLP-2 and short bowel syndrome (SBS). Several studies in animals and from clinical trials have shown that GLP-2 is a trophic hormone that plays an important role in intestinal adaptation. Its role in regulation of cell proliferation, apoptosis, and nutrient absorption has been well documented. Short bowel syndrome is characterized by malabsorption of nutrients, water and vitamins as a result of disease or surgical removal of parts of the small intestine (eg. Crohn's disease). Therapies that improve intestinal adaptation are thought to be beneficial in treatment of this disease. In fact, phase II studies in SBS patients have shown that teduglutide, a GLP-2 analog, modestly increased fluid and nutrient absorption.

In an embodiment of the invention are therapeutic benefits of GPR119 activity modulators derived from increasing levels of GIP and PPY. For example, GLP-1, GIP and osteoporosis. GLP-1 has been shown to increase calcitonin and calcitonin related gene peptide (CGRP) secretion and expression in a murine C-cell line (CA-77). Calcitonin inhibits bone resorption by osteoclasts and promotes mineralization of skeletal bone. Osteoporosis is a disease that is characterized by reduced bone mineral density and thus GLP-1 induced increase in calcitonin might be therapeutically beneficial.

GIP has been reported to be involved in upregulation of markers of new bone formation in osetoblasts including collagen type I mRNA and in increasing bone mineral density. Like GLP-1, GIP has also been shown to inhibit bone resorption.

In an embodiment of the invention are therapeutic benefits of GPR119 activity modulators derived from increasing levels of GIP and PPY. For example, PPY and gastric emptying. GPR119 located on the pancreatic polypeptide (PP) cells of the islets has been implicated in the secretion of PPY. PPY has been reported to have profound effects on various physiological processes including modulation of gastric emptying and gastrointestinal motility. These effects slow down the digestive process and nutrient uptake and thereby prevent the postprandial elevation of blood glucose. PPY can suppress food intake by changing the expression of hypothalamic feeding-regulatory peptides. PP-overexpressing mice exhibited the thin phenotype with decreased food intake and gastric emptying rate.

In accordance with the foregoing, the present invention further provides a method for preventing or ameliorating the symptomology of any of the diseases or disorders described above in a subject in need thereof, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrollidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

Compounds of the invention can be administered in therapeutically effective amounts in combination with one or more therapeutic agents (pharmaceutical combinations).

For example, synergistic effects can occur with other anti-obesity agents, anorectic agents, appetite suppressant and related agents. Diet and/or exercise can also have synergistic effects. Anti-obesity agents include, but are not limited to, apolipoprotein-B secretion/microsomal triglyceride transfer protein (apo-B/MTP) inhibitors, MCR-4 agonists, cholescystokinin-A (CCK-A) agonists, serotonin and norepinephrine reuptake inhibitors (for example, sibutramine), sympathomimetic agents, β3 adrenergic receptor agonists, dopamine agonists (for example, bromocriptine), melanocyte-stimulating hormone receptor analogs, cannabinoid 1 receptor antagonists [for example, compounds described in WO02006/047516), melanin concentrating hormone antagonists, leptons (the OB protein), leptin analogues, leptin receptor agonists, galanin antagonists, lipase inhibitors (such as tetrahydrolipstatin, i.e., Orlistat), anorectic agents (such as a bombesin agonist), Neuropeptide-Y antagonists, thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™), human agouti-related proteins (AGRP), ghrelin receptor antagonists, histamine 3 receptor antagonists or reverse agonists, neuromedin U receptor agonists, noradrenergic anorectic agents (for example, phentermine, mazindol and the like) and appetite suppressants (for example, bupropion).

Where compounds of the invention are administered in conjunction with other therapies, dosages of the co-administered compounds will of course vary depending on the type of co-drug employed, on the specific drug employed, on the condition being treated and so forth.

A combined preparation or pharmaceutical composition can comprise a compound of the invention as defined above or a pharmaceutical acceptable salt thereof and at least one active ingredient selected from:

a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; DPPIV (dipeptidyl peptidase IV) inhibitors such as DPP728, LAF237 (vildagliptin—Example 1 of WO 00/34241), MK-0431, saxagliptin, GSK23A; an AGE breaker; a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid described in the patent application WO 03/043985, as compound 19 of Example 4, a non-glitazone type PPAR gamma agonist e.g. GI-262570; Diacylglycerol acetyltransferase (DGAT) inhibitors such as those disclosed in WO 2005044250, WO 2005013907, WO 2004094618 and WO 2004047755;

b) hypolipidemic agents such as 3-hydroxy-3-methyl-glutaryl coenzyme A (HMG-CoA) reductase inhibitors, e.g., lovastatin and related compounds such as those disclosed in U.S. Pat. No. 4,231,938, pitavastatin, simvastatin and related compounds such as those disclosed in U.S. Pat. Nos. 4,448,784 and 4,450,171, pravastatin and related compounds such as those disclosed in U.S. Pat. No. 4,346,227, cerivastatin, mevastatin and related compounds such as those disclosed in U.S. Pat. No. 3,983,140, velostatin, fluvastatin, dalvastatin, atorvastatin, rosuvastatin and related statin compounds disclosed in U.S. Pat. No. 5,753,675, rivastatin, pyrazole analogs of mevalonolactone derivatives as disclosed in U.S. Pat. No. 4,613,610, indene analogs of mevalonolactone derivatives as disclosed in PCT application WO 86/03488, 6-[2-(substituted-pyrrol-1-yl)-alkyl)pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054, 3-carboxy-2-hydroxy-propane-phosphonic acid derivatives as disclosed in French Patent No. 2,596,393, 2,3-disubstituted pyrrole, furan and thiophene derivatives as disclosed in European Patent Application No. 0221025, naphthyl analogs of mevalonolactone as disclosed in U.S. Pat. No. 4,686,237, octahydronaphthalenes such as disclosed in U.S. Pat. No. 4,499,289, keto analogs of mevinolin (lovastatin) as disclosed in European Patent Application No. 0,142,146 A2, and quinoline and pyridine derivatives disclosed in U.S. Pat. Nos. 5,506,219 and 5,691,322. In addition, phosphinic acid compounds useful in inhibiting HMG CoA reductase suitable for use herein are disclosed in GB 2205837; squalene synthase inhibitors; FXR (farnesoid X receptor) and LXR (liver X receptor) ligands; cholestyramine; fibrates; nicotinic acid and aspirin;

c) an anti-obesity agent or appetite regulating agent such as a CB1 activity modulator, melanocortin receptor (MC4R) agonists, melanin-concentrating hormone receptor (MCHR) antagonists, growth hormone secretagogue receptor (GHSR) antagonists, galanin receptor modulators, orexin antagonists, CCK agonists, GLP-1 agonists, and other Pre-proglucagon-derived peptides; NPY1 or NPY5 antagonist, NPY2 and NPY4 modulators, corticotropin releasing factor agonists, histamine receptor-3 (H3) modulators, aP2 inhibitors, PPAR gamma modulators, PPAR delta modulators, acetyl-CoA carboxylase (ACC) inhibitors, 11-β-HSD-1 inhibitors, adinopectin receptor modulators; beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5, 491,134, 5,776,983 and 5,488,064, a thyroid receptor beta modulator, such as a thyroid receptor ligand as disclosed in WO 97/21993 (U. Cal SF), WO 99/00353 (KaroBio) and GB98/284425 (KaroBio), a SCD-1 inhibitor as disclosed in WO2005011655, a lipase inhibitor, such as orlistat or ATL-962 (Alizyme), serotonin receptor agonists, (e.g., BVT-933 (Biovitrum)), monoamine reuptake inhibitors or releasing agents, such as fenfluramine, dexfenfluramine, fluvoxamine, fluoxetine, paroxetine, sertraline, chlorphentermine, cloforex, clortermine, picilorex, sibutramine, dexamphetamine, phentermine, phenylpropanolamine or mazindol, anorectic agents such as topiramate (Johnson & Johnson), CNTF (ciliary neurotrophic factor)/Axokine® (Regeneron), BDNF (brain-derived neurotrophic factor), leptin and leptin receptor modulators, phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine;

d) anti-hypertensive agents such as loop diuretics such as ethacrynic acid, furosemide and torsemide; diuretics such as thiazide derivatives, chlorithiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; beta-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; aldosterone synthase inhibitors; and dual ET/AII antagonist such as those disclosed in WO 00/01389.

e) a HDL increasing compound;

f) Cholesterol absorption modulator such as Zetia® and KT6-971;

g) Apo-A1 analogues and mimetics;

h) thrombin inhibitors such as Ximelagatran;

i) aldosterone inhibitors such as anastrazole, fadrazole, eplerenone;

j) Inhibitors of platelet aggregation such as aspirin, clopidogrel bisulfate;

k) estrogen, testosterone, a selective estrogen receptor modulator, a selective androgen receptor modulator;

l) a chemotherapeutic agent such as aromatase inhibitors e.g. femara, anti-estrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib ({N-{5-[4-(4-methyl-piperazino-methyl)-benzoylamido]-2-methylphenyl}-4-(3-pyridyl)-2-pyrimidine-amine}) described in the European patent application EP-A-0 564 409 as example 21 or 4-Methyl-N-[3-(4-methyl-imidazol-1-yl)-5-trifluoromethyl-phenyl]-3-(4-pyridin-3-yl-pyrimidin-2-ylamino)-benzamide described in the patent application WO 04/005281 as example 92; and

m) an agent interacting with a 5-HT₃ receptor and/or an agent interacting with 5-HT₄ receptor such as tegaserod described in the U.S. Pat. No. 5,510,353 as example 13, tegaserod hydrogen maleate, cisapride, cilansetron;

n) an agent for treating tobacco abuse, e.g., nicotine receptor partial agonists, bupropion hypochloride (also known under the tradename Zyban®) and nicotine replacement therapies;

o) an agent for treating erectile dysfunction, e.g., dopaminergic agents, such as apomorphine), ADD/ADHD agents (e.g., Ritalin®, Strattera®, Concerta® and Adderall®);

p) an agent for treating alcoholism, such as opioid antagonists (e.g., naltrexone (also known under the tradename ReVia®) and nalmefene), disulfiram (also known under the tradename Antabuse®), and acamprosate (also known under the tradename Campral®)). In addition, agents for reducing alcohol withdrawal symptoms may also be co-administered, such as benzodiazepines, beta-blockers, clonidine, carbamazepine, pregabalin, and gabapentin (Neurontin®);

q) other agents that are useful including anti-inflammatory agents (e.g., COX-2 inhibitors); antidepressants (e.g., fluoxetine hydrochloride (Prozac®)); cognitive improvement agents (e.g., donepezil hydrochloride (Aircept®) and other acetylcholinesterase inhibitors); neuroprotective agents (e.g., memantine); antipsychotic medications (e.g., ziprasidone (Geodon®), risperidone (Risperdal®), and olanzapine (Zyprexa®));

or, in each case a pharmaceutically acceptable salt thereof; and optionally a pharmaceutically acceptable carrier.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

In the following schemes, several methods of preparing the compounds of the present invention are illustrative. One of skill in the art will appreciate that these methods are representative, and in no way inclusive of all methods for preparing the compounds of the present invention. The radicals in the schemes are as described in Formula I.

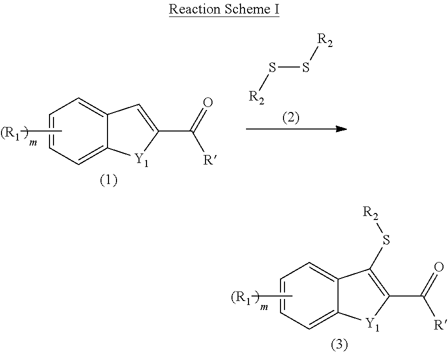

A compound of Formula 3 can be prepared by reacting a compound of formula 1 with a dithiane of formula 2 (R2=aromatic), in the presence of a suitable solvent (for example, dimethylformamide, and the like) and a suitable base (for example, cesiumcarbonate, and the like). The reaction proceeds at a temperature of about 50° C. to about 150° C. and can take up to 10 h to complete.

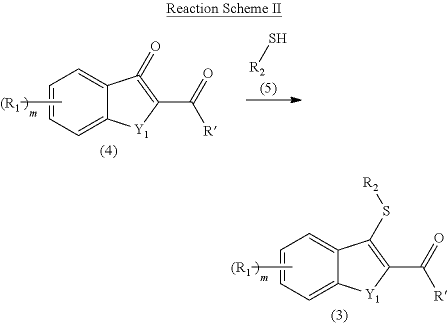

A compound of Formula 3 can be prepared by reacting a compound of formula 4 with a thiol of formula 5 (R2=aromatic), neat and in the presence of a suitable acid (for example, polyphosphoric acid, and the like). The reaction proceeds at a temperature of about 50° C. to about 150° C. and can take up to 5 h to complete.

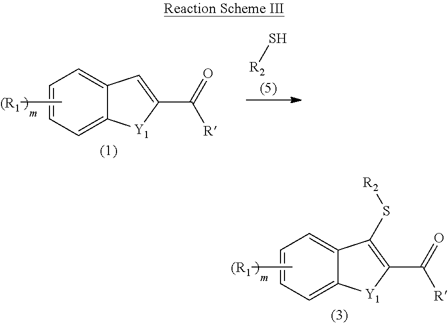

A compound of Formula 3 can be prepared by reacting a compound of formula 1 with a thiol of formula 2 (R2=aliphatic), in the presence of a suitable solvent (for example, dichloromethane, and the like) and a suitable activator (for example, N-chlorosuccinimide, and the like). The reaction proceeds at a temperature of about −10° C. to about 25° C. and can take up to 5 h to complete.

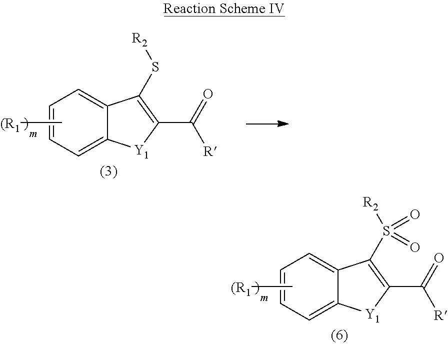

A compound of Formula 6 can be prepared by reacting a compound of formula 3 in the presence of a suitable solvent (for example, chloroform, and the like) and a suitable oxidant (for example, meta-chloroperbenzoic acid, and the like). The reaction proceeds at a temperature of about 0° C. to about 50° C. and can take up to 10 h to complete.

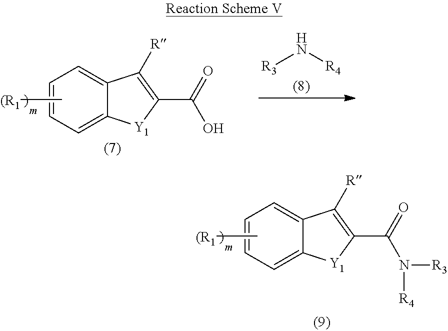

A compound of Formula 9 can be prepared by reacting a compound of formula 7 with an amine of formula 8, in the presence of a suitable solvent (for example, dimethylformamide, tetrahydrofuran, and the like), a suitable base (for example, diisopropylethylamine, and the like) and a suitable coupling reagent (for example, HATU, carbonyldiimidazole, and the like). The reaction proceeds at a temperature of about 0° C. to about 50° C. and can take up to 24 h to complete.

Detailed descriptions of the synthesis of compounds of the Invention are given in the Examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction schemes I, II, III, IV & V; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following Examples that illustrate the preparation of compounds of the invention and their intermediates.

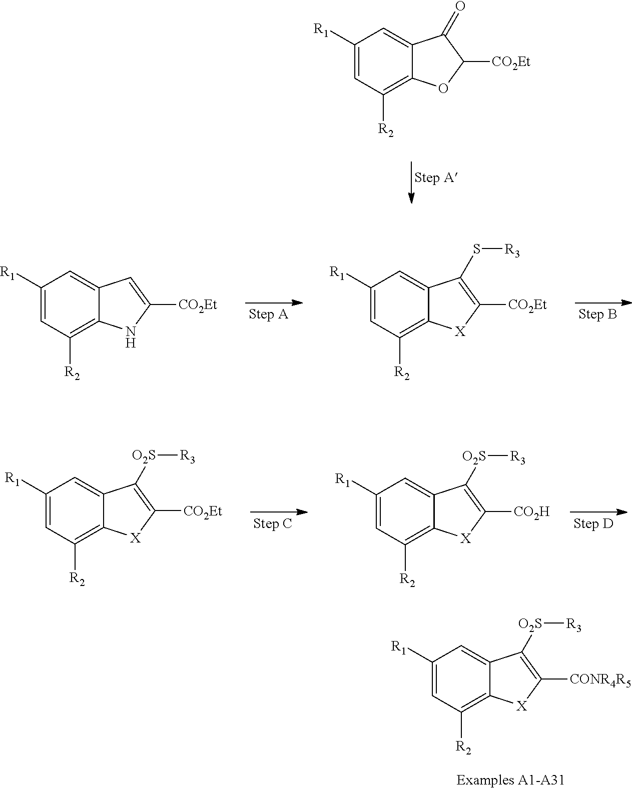

### Example A1

### (4-benzylpiperidin-1-yl)(3-(4-chlorophenylsulfonyl)-5-fluoro-1H-indol-2-yl)methanone

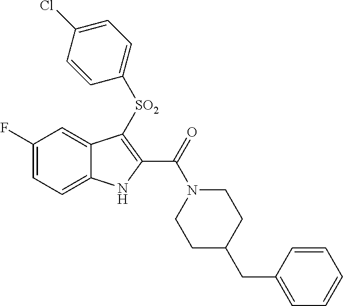

Step A (R₃=aryl): A mixture of ethyl 5-fluoro-1H-indole-2-carboxylate (311 mg, 1.5 mmol), 1,2-bis(4-chlorophenyl)disulfane (645 mg, 2.25 mmol) and Cs₂CO₃ (587 mg, 1.8 mmol) in DMF (20 mL) is stirred at 100° C. for 2.5 h under a N₂ atmosphere. After cooling to rt, H₂O is added to the reaction mixture and the product is extracted with DCM (3×20 mL). The organic layers are combined, dried (Na₂SO₄), filtered and concentrated. The mixture is purified by flash chromatography on silica gel (hexanes/EtOAc gradient) to afford ethyl 3-(4-chlorophenylthio)-5-fluoro-1H-indole-2-carboxylate. ¹H-NMR (400 MHz, DMSO-d6) δ=12.60 (s, 1H), 7.57 (dd, 1H, J=4.5, 9.0 Hz), 7.29 (m, 2H), 7.23 (td, 1H, J=2.6, 9.2 Hz), 7.15 (dd, J=2.6, J=9.3 Hz), 7.07 (m, 2H), 4.32 (q, 2H, J=7.1 Hz), 1.23 (t, 3H, J=7.1 Hz). MS calcd. for C₁₇H₁₄ClFNO₂S (M+H⁺) 350.0, found 350.0.

Step B. MCPBA (70-75%, 943 mg, 3.82 mmol) is added to a cold (ice bath) solution of ethyl 3-(4-chlorophenylthio)-5-fluoro-1H-indole-2-carboxylate (446 mg, 1.27 mmol) in CHCl₃ (22 mL) and stirred at rt for 2.5 h. Saturated aqu. NaHCO₃ is added and the mixture is extracted with CHCl₃ (3×20 mL). The organic layers are combined, dried (Na₂SO₄) and concentrated to give crude ethyl 3-(4-chlorophenylsulfonyl)-5-fluoro-1H-indole-2-carboxylate. ¹H-NMR (400 MHz, DMSO-d6) δ=8.02 (m, 2H), 7.92 (dd, 1H, J=2.6, 10.1 Hz), 7.67 (m, 2H), 7.62 (dd, 1H, J=4.6, 9.1 Hz), 7.33 (td, 1H, J=2.6, 9.2 Hz), 4.36 (q, 2H, J=7.1 Hz), 1.30 (t, 3H, J=7.1 Hz). MS calcd. for C₁₇H₁₄ClFNO₄S (M+H⁺) 382.0, found 382.0.

Step C. Crude ethyl 3-(4-chlorophenylsulfonyl)-5-fluoro-1H-indole-2-carboxylate (˜1.2 mmol) is dissolved in THF/MeOH 1:1 (12 mL), then 2N NaOH (6 mL, 12 mmol) is added. The mixture is stirred at rt overnight, then concentrated in vacuo. H₂O is added and the solution is filtered. The filtrate is acidified with 1N HCl and the precipitated 3-(4-chlorophenylsulfonyl)-5-fluoro-1H-indole-2-carboxylic acid is filtered, washed with H₂O and dried under high vacuum. ¹H-NMR (400 MHz, DMSO-d6) ≢=12.22 (bs, 1H), 8.20 (m, 2H), 7.71 (dd, 1H, J=2.6, 10.5 Hz), 7.54 (m, 2H), 7.39 (dd, 1H, J=4.8, 8.9 Hz), 7.03 (td, 1H, J=2.6, 9.1 Hz). MS calcd. for C₁₅H₁₀ClFNO₄S (M+H⁺) 354.0, found 354.0.

Step D. To a solution of 3-(4-chlorophenylsulfonyl)-5-fluoro-1H-indole-2-carboxylic acid (17.7 mg, 0.05 mmol) and 4-benzylpiperidine (10.5 mg, 0.06 mmol) in DMF (0.4 mL) is added HATU (22.8 mg, 0.06 mmol) and DIEA (26 μL, 0.15 mmol). The mixture is stirred at rt overnight. The title compound is obtained after purification by reverse phase HPLC (H₂O/MeCN gradient). ¹H-NMR (400 MHz, DMSO-d6) δ=8.05 (d, 2H, J=8.4 Hz), 7.66 (m, 2H), 7.52 (m, 2H), 7.28 (m, 2H), 7.17 (m, 4H), 4.53 (d, 1H, J=13.0 Hz), 3.25 (m, 1H), 3.03 (m, 1H), 2.81 (m, 1H), 2.52 (m, 2H), 1.84 (m, 1H), 1.70 (m, 1H), 1.52 (m, 1H), 1.15-1-40 (m, 2H). MS calcd. for C₂₇H₂₅ClFN₂O₃S (M+H⁺) 511.1, found 511.1.

### Example A29

### (3-(4-Chlorophenylsulfonyl)benzofuran-2-yl)(4-(4-(trifluoromethyl)phenoxy)piperidin-1-yl)methanone

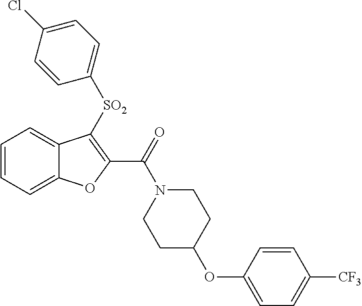

Step A′: Polyphosphoric acid (7 g) is added to a mixture of 4-chlorobenzenethiol (289 mg, 2 mmol) and ethyl 3-oxo-2,3-dihydrobenzofuran-2-carboxylate (495 mg, 2.4 mmol) and stirred at 95° C. for 1.5 h. Ice-water (75 mL) is added to the reaction mixture and the product is extracted with EtOAc (3×30 mL). The organic layers are combined, dried (Na₂SO₄), filtered and concentrated. The mixture is purified by flash chromatography on silica gel (hexanes/EtOAc gradient) to obtain ethyl 3-(4-chlorophenylthio)benzofuran-2-carboxylate. MS calcd. for C₁₇H₁₄ClO₃S (M+H⁺) 332.0, found 332.0.

Step B-D: Steps B-D are performed according to steps B-D for example A1 and afforded (3-(4-chlorophenylsulfonyl)benzofuran-2-yl)(4-(4-(trifluoromethyl)phenoxy)piperidin-1-yl) A29. ¹H-NMR (400 MHz, DMSO-d6) δ=8.15 (m, 2H), 7.90 (m, 1H), 7.81 (d, 1H, J=8.2 Hz), 7.76 (m, 2H), 7.66 (m, 2H), 7.55 (m, 1H), 7.49 (m, 1H), 7.21 (m, 2H), 4.89 (m, 1H), 4.04 (m, 1H), 3.63 (m, 1H), 3.54 (m, 1H), 3.38 (m, 1H), 2.07 (m, 2H), 1.75 (m, 2H). MS calcd. for C₂₇H₂₂ClF₃NO₅S (M+H⁺) 564.1, found 564.0.

By repeating the procedure described in the above examples A1 and A29, using appropriate starting materials, the following compounds of Formula I, as identified in Table 1, are obtained.

TABLE 1

| | | Physical Data |
|---|---|---|
| Compound | Compound | ¹H NMR 400 MHz (DMSO-d₆) |
| Number | Structure | and/or MS (m/z) |
| A2 | 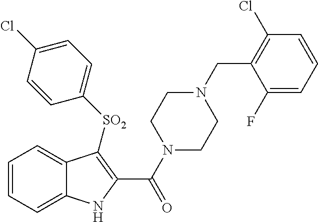 | ¹H-NMR (400 MHz, DMSO-d6) δ = 12.84 (s, 1H), 8.02 (m, 2H), 7.83 (d, 1H, J = 7.8 Hz), 7.66 (m, 2H), 7.53 (m, 2H), 7.46 (d, 1H, 7.7 Hz), 7.25-7.39 (m, 3H), 2.7-4.5 (m, 10H). MS calcd. for C₂₆H₂₃Cl₂FN₃O₃S (M + H⁺) 546.1, found 546.0. |
| A3 | 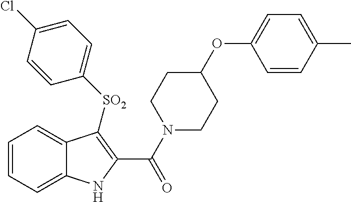 | ¹H-NMR (400 MHz, DMSO-d6) δ = 12.84 (s, 1H), 8.05 (m, 2H), 7.82 (d, 1H, J = 7.7 Hz), 7.66 (m, 2H), 7.50 (d, 1H, J = 7.8 Hz), 7.31 (m, 1H), 7.26 (m, 1H), 7.08 (m, 2H), 6.90 (m, 2H), 4.66 (m, 1H), 4.06 (bs, 1H), 3.57 (bs, 1H), 3.39 (m, 1H), 3.22 (m, 1H), 2.22 (s, 3H), 2.00 (m, 2H), 1.72 (m, 2H). MS calcd. for C₂₇H₂₆ClN₂O₄S (M + H⁺) 509.1, found 509.3. |
| A4 | 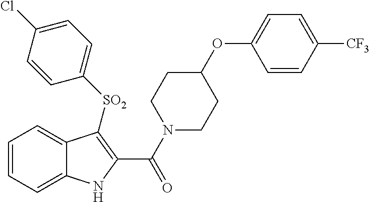 | ¹H-NMR (400 MHz, DMSO-d6) δ = 12.84 (s, 1H), 8.05 (m, 2H), 7.83 (d, 1H, J = 7.7 Hz), 7.66 (m, 4H), 7.50 (d, 1H, J = 7.8 Hz), 7.31 (m, 1H), 7.26 (m, 1H), 7.20 (d, 2H, J = 8.6 Hz), 4.88 (m, 1H), 4.09 (bs, 1H), 3.59 (m, 1H), 3.40 (m, 1H), 3.26 (m, 1H), 2.05 (m, 2H), 1.77 (m, 2H). MS calcd. for C₂₇H₂₃ClF₃N₂O₄S (M + H⁺) 563.1, found 563.0. |
| A5 | 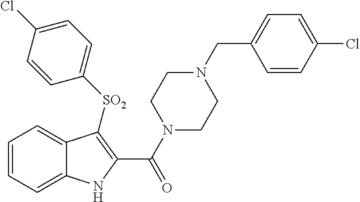 | ¹H-NMR (400 MHz, DMSO-d6) δ = 12.88 (s, 1H), 10.29 (bs, 1H), 8.02 (d, 2H, J = 8.5 Hz), 7.84 (d, 1H, J = 7.8 Hz), 7.67 (m, 2H), 7.54 (m, 5H), 7.33 (m, 1H), 7.28 (m, 1H), 2.7-4.8 (m, 10H). MS calcd. for C₂₆H₂₄Cl₂N₃O₃S (M + H⁺) 528.1, found 528.0. |
| A6 | 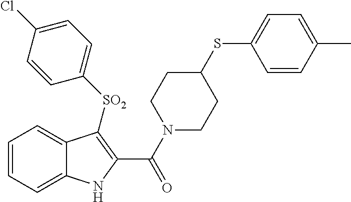 | ¹H-NMR (400 MHz, DMSO-d6) δ = 12.81 (s, 1H), 8.03 (d, 2H, J = 8.6 Hz), 7.81 (d, 1H, J = 7.7 Hz), 7.65 (m, 2H), 7.48 (d, 1H, J = 7.9), 7.34 (d, 2H, J = 8.1 Hz), 7.30 (m, 1H), 7.25 (m, 1H), 7.17 (d, 2H, J = 8.0 Hz), 4.40 (d, 1H, J = 12.6 Hz), 3.50 (m, 1H), 3.31 (m, 1H), 3.15 (m, 2H), 2.28 (s, 3H), 2.01 (m 1H), 1.85 (m, 1H), 1.45-1.70 (m, 2H). MS calcd. for C₂₇H₂₆ClN₂O₃S₂ (M + H⁺) 525.1, found 525.2. |
| A7 | 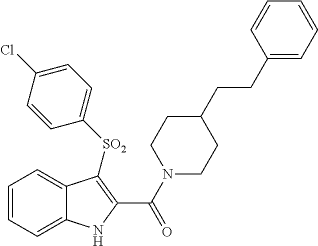 | ¹H-NMR (400 MHz, DMSO-d6) δ = 12.81 (s, 1H),. 8.04 (d, 2H, J = 8.4 Hz), 7.83 (d, 1H, J = 7.7 Hz), 7.65 (m, 2H), 7.48 (d, 1H, J = 7.7 Hz), 7.14-7.33 (m, 7H), 4.56 (d, 1H, 12.8 Hz), 3.27 (d, 1H, J = 13.1 Hz), 3.06 (m, 1H), 2.84 (m, 1H), 2.61 (t, 2H, j = 7.6 Hz), 1.86 (m, 1H) 1.65 (m, 1H), 1.54 (m, 3H), 1.25 (m, 2H). MS calcd. for C₂₈H₂₈ClN₂O₃S (M + H⁺) 507.1, found 507.3. |
| A8 | 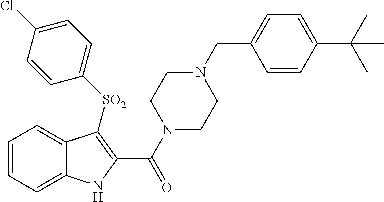 | ¹H-NMR (400 MHz, DMSO-d6) δ = 12.88 (s, 1H), 10.09 (bs, 1H), 8.02 (d, 2H, J = 8.6 Hz), 7.84 (d, 1H, J = 7.8 Hz), 7.67 (m, 2H), 7.49 (m, 5H), 7.33 (m, 1H), 7.26 (m, 1H), 4.67 (m, 1H), 4.38 (m, 2H), 2.9-3.7 (m, 7H), 1.29 (s, 9H). MS calcd. for C₃₀H₃₃ClN₃O₃S (M + H⁺) 550.2, found 550.1. |
| A9 | 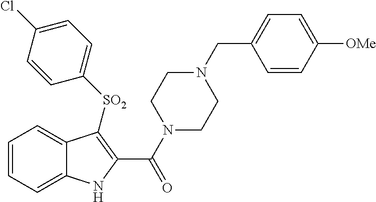 | ¹H-NMR (400 MHz, DMSO-d6) δ = 12.92 (s, 1H), 10.22 (bs, 1H), 8.02 (d, 2H, J = 8.5 Hz), 7.84 (d, 1H, J = 7.8 Hz), 7.67 (m, 2H), 7.54 (d, 1H, 8.0 Hz), 7.46 (d, 2H, J = 8.5 Hz), 7.33 (m, 1H), 7.28 (m, 1H), 7.03 (d, 2H, 8.6 Hz), 4.67 (m, 1H), 4.34 (bs, 2H), 3.78 (s, 3H), 2.9-3.7 (m, 7H). MS calcd. for C₂₇H₂₇ClN₃O₄S (M + H⁺) 524.1, found 524.2. |
| A10 | 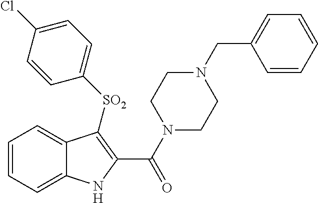 | ¹H-NMR (400 MHz, DMSO-d6) δ = 12.91 (s, 1H), 10.30 (bs, 1H), 8.02 (d, 2H, J = 8.6 Hz), 7.84 (d, 1H, J = 7.8 Hz), 7.67 (m, 2H), 7.50 (m, 6H), 7.33 (m, 1H), 7.26 (m, 1H), 2.8-4.9 (m, 10H). MS calcd. for C₂₆H₂₅ClN₃O₃S (M + H⁺) 494.1, found 494.3. |
| A11 | 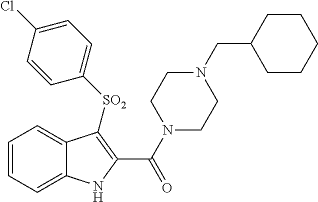 | ¹H-NMR (400 MHz, DMSO-d6) δ = 12.97 (s, 1H), 9.75 (bs, 1H), 8.03 (d, 2H, J = 8.6 Hz), 7.85 (d, 1H, J = 7.8 Hz), 7.67 (m, 2H), 7.54 (d, 1H, J = 8.0 Hz), 7.34 (m, 1H), 7.28 (m, 1H), 4.62 (m, 1H), 3.69 (m, 1H), 3.47 (m, 4H), 3.04 (bs, 4H), 1.64 (m, 6H), 1.19 (m, 3H), 0.96 (m, 2H). MS calcd. for C₂₆H₃₁ClN₃O₃S (M + H⁺) 501.2, found 501.3. |
| A12 | 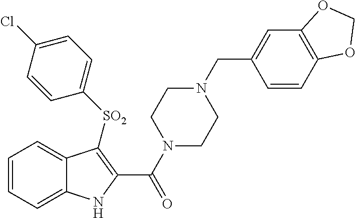 | ¹H-NMR (400 MHz, DMSO-d6) δ = 12.92 (s, 1H), 10.23 (bs, 1H), 8.03 (d, 2H, J = 8.5 Hz), 7.84 (d, 1H, J = 7.9 Hz), 7.67 (m, 2H), 7.54 (d, 1H, J = 7.9 Hz), 7.33 (m, 1H), 7.28 (m, 1H), 7.12 (s, 1H), 7.02 (s, 2H), 6.08 (s, 2H), 4.67 (bs, 1H), 4.31 (bs, 2H), 2.9-3.7 (m, 7H). MS calcd. for C₂₇H₂₅ClN₃O₅S (M + H⁺) 538.1, found 538.2. |
| A13 | 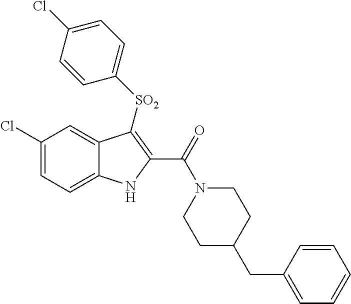 | ¹H-NMR (400 MHz, DMSO-d6) δ = 8.05 (d, 2H, J = 8.4 Hz), 7.79 (d, 1H, J = 2.0 Hz), 7.67 (m, 2H), 7.52 (d, 1H, J = 8.7 Hz), 7.33 (dd, 1H, J = 2.1, 8.7 Hz), 7.28 (m, 2H), 7.18 (m, 3H), 4.53 (m, 1H), 3.24 (m, 1H), 3.03 (m, 1H), 2.81 (m, 1H), 2.52 (m, 2H), 1.84 (m, 1H), 1.71 (m, 1H), 1.52 (m, 1H), 1.25 (m, 2H). MS calcd. for C₂₇H₂₅Cl₂N₂O₃S (M + H⁺) 527.1, found 527.1. |
| A14 | 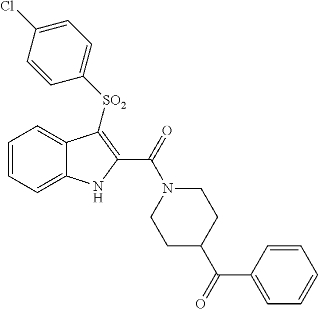 | ¹H-NMR (400 MHz, DMSO-d6) δ = 12.81 (s, 1H), 8.03 (m, 4H), 7.82 (d, 1H, J = 7.7 Hz), 7.65 (m, 3H), 7.55 (m, 2H), 7.49 (d, 1H, 7.9 Hz), 7.30 (m, 1H), 7.25 (m, 1H), 4.60 (m, 1H), 3.83 (m, 1H), 3.29 (m, 2H), 3.13 (m, 1H), 1.96 (m, 1H), 1.69 (m, 3H). MS calcd. for C₂₇H₂₄ClN₂O₄S (M + H⁺) 507.1, found 507.2. |
| A15 | 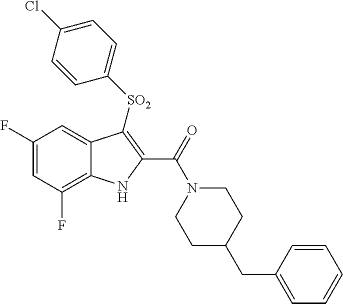 | ¹H-NMR (400 MHz, DMSO-d6) δ = 8.06 (m, 2H), 7.67 (d, 2H, J = 8.4 Hz), 7.42 (d, 1H, J = 7.9 Hz), 7.28 (m, 3H), 7.17 (m, 3H), 4.53 (m, 1H), 3.27 (m, 1H), 3.03 (m, 1H), 2.81 (m, 1H), 2.52 (m, 2H), 1.84 (m, 1H), 1.70 (m, 1H), 1.51 (m, 1H), 1.15-1.40 (m, 2H). MS calcd. for C₂₇H₂₄ClF₂N₂O₃S (M + H⁺) 529.1, found 529.1. |
| A16 | 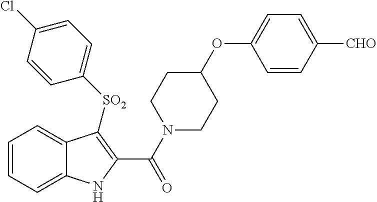 | ¹H-NMR (400 MHz, DMSO-d6) δ = 12.85 (s, 1H), 9.87 (s, 1H), 8.05 (m, 2H), 7.87 (m, 2H), 7.83 (d, 1H, J = 7.7 Hz), 7.66 (m, 2H), 7.50 (d, 1H, J = 7.8 Hz), 7.31 (m, 1H), 7.26 (m, 1H), 7.21 (m, 2H), 4.93 (m, 1H), 4.11 (m, 1H), 3.59 (m, 1H), 3.39 (m, 1H), 3.27 (m, 1H), 2.11 (m, 1H), 2.02 (m, 1H), 1.78 (m, 2H). MS calcd. for C₂₇H₂₄ClN₂O₅S (M + H⁺) 523.1, found 523.2. |
| A17 | 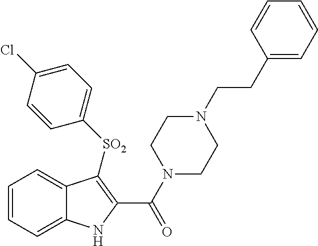 | ¹H-NMR (400 MHz, DMSO-d6) δ = 12.97 (s, 1H), 10.41 (bs, 1H), 8.04 (d, 2H, J = 8.6 Hz), 7.86 (d, 1H, J = 7.8 Hz), 7.68 (m, 2H), 7.55 (d, 1H, J = 8.0 Hz), 7.31 (m, 7H), 4.69 (m, 1H), 2.9-4.0 (m, 11H). MS calcd. for C₂₇H₂₇ClN₃O₃S (M + H⁺) 508.1, found 508.1. |
| A18 | 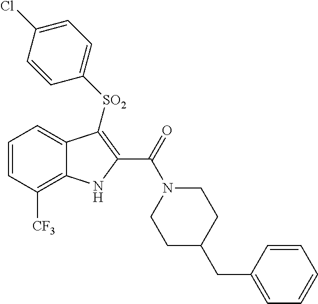 | ¹H-NMR (400 MHz, DMSO-d6) δ = 8.15 (m, 1H), 8.05 (m, 2H), 7.67 (m, 3H), 7.44 (t, 1H, J = 7.7 Hz), 7.28 (m, 2H), 7.19 (m, 3H), 4.55 (m, 1H), 3.36 (m, 1H), 3.24 (m, 1H), 3.03 (m, 1H), 2.80 (m, 1H), 2.56 (m, 1H), 1.84 (m, 1H), 1.69 (m, 1H), 1.51 (m, 1H), 1.28-1.42 (m, 2H). MS calcd. for C₂₈H₂₅ClF₃N₂O₃S (M + H⁺) 561.1, found 561.1. |
| A19 | 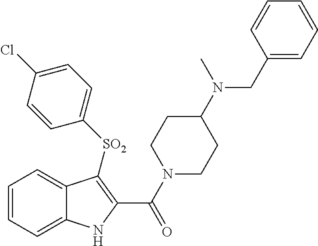 | ¹H-NMR (400 MHz, DMSO-d6) δ = 12.93 (s, 1H), 9.71 (m, 1H), 8.05 (d, 2H, J = 8.2 Hz), 7.84 (d, 1H, J = 7.8 Hz), 7.67 (m, 2H), 7.52 (m, 6H), 7.30 (m, 2H), 4.75 (m, 1H), 4.47 (m, 1H), 4.24 (m, 1H), 3.69 (m, 1H), 3.45 (m, 1H), 3.21 (m, 1H), 2.93 (m, 1H), 2.62 (d, 3H, J = 4.6 Hz), 2.25 (m, 1H), 1.80 (m, 2H). MS calcd. for C₂₈H₂₉ClN₃O₃S (M + H⁺) 522.2, found 522.3. |
| A20 | 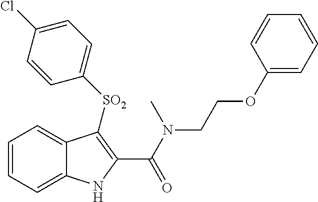 | ¹H-NMR (400 MHz, DMSO-d6) 54:46 rotamer ratio, δ = 12.87 (s, 0.46H), 12.85 (s 0.54H), 8.02 (m, 2H), 7.84 (d, 1H, J = 7.9 Hz), 7.65 (m, 2H), 7.50 (m, 1H), 7.27 (m, 4H), 7.02 (m, 1H), 6.97 (m, 0.54H), 6.89 (m, 1.46H), 4.29 (t, 1.08 H, J = 5.6 Hz), 4.14 (m, 0.92H), 3.94 (t, 1.08 H, J = 5.6 Hz), 3.60 (m, 0.92H), 3.21 (s, 1.38H), 2.99 (s, 1.62H). MS calcd. for C₂₄H₂₂ClN₂O₄S (M + H⁺) 469.1, found 469.2. |
| A21 | 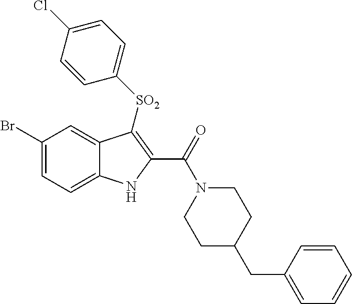 | ¹H-NMR (400 MHz, DMSO-d6) δ = 8.04 (d, 2H, J = 8.4 Hz)), 7.93 (d, 1H, J = 1.6 Hz), 7.67 (m, 2H), 7.47 (d, 1H, J = 8.6 Hz), 7.44 (dd, 1H, J = 1.8, 8.7 Hz), 7.28 (m, 2H), 7.18 (m, 3H), 4.52 (m, 1H), 3.23 (m, 1H), 3.02 (m, 1H), 2.81 (m, 1H), 2.52 (m, 2H), 1.84 (m, 1H), 1.71 (m, 1H), 1.51 (m, 1H), 1.13-1.36 (m, 2H). MS calcd. for C₂₇H₂₅BrClN₂O₃S (M + H⁺) 571.0, found 571.0. |
| A22 | 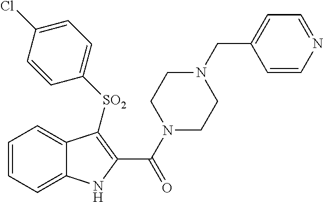 | ¹H-NMR (400 MHz, DMSO-d6) δ = 12.92 (s, 1H), 8.82 (d, 2H, J = 6.3 Hz), 8.03 (m, 2H), 7.83 (m, 3H), 7.67 (m, 2H), 7.52 (d, 1H, J = 7.9 Hz), 7.32 (m, 1H), 7.27 (m, 1H), 4.27 (s, 2H), 3.90 (bs, 2H), 3.44 (m, 2H), 2.99 (m, 4H). MS calcd. for C₂₅H₂₄ClN₄O₃S (M + H⁺) 495.1, found 495.1. |
| A23 | 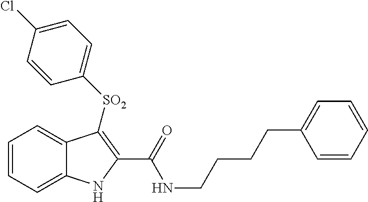 | ¹H-NMR (400 MHz, DMSO-d6) δ = 12.81 (s, 1H), 8.93 (t, 1H, J = 5.5 Hz), 8.03 (m, 2H), 7.94 (d, 1H, J = 7.6 Hz), 7.62 (m, 2H), 7.50 (d, 1H, J = 7.9 Hz), 7.21-7.34 (m, 6H), 7.17 (m, 1H), 3.37 (m, 2H), 2.64 (t, 2H, J = 7.5 Hz), 1.71 (m, 2H), 1.61 (m, 2H). MS calcd. for C₂₅H₂₄ClN₂O₃S (M + H⁺) 467.1, found 467.1. |
| A24 | 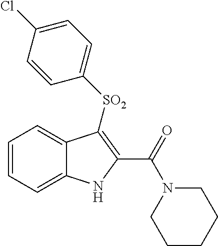 | ¹H-NMR (400 MHz, DMSO-d6) δ = 12.78 (s, 1H), 8.04 (d, 2H), 7.82 (d, 1H, J = 7.4 Hz), 7.65 (m, 2H), 7.48 (d, 1H, J = 7.7 Hz), 7.30 (m, 1H), 7.25 (m, 1H), 3.68 (m, 2H), 3.16 (m, 2H, J = 5.3 Hz), 1.64 (s, 4H), 1.55 (s, 2H). MS calcd. for C₂₀H₂₀ClN₂O₃S (M + H⁺) 403.1, found 403.1. |
| A25 | 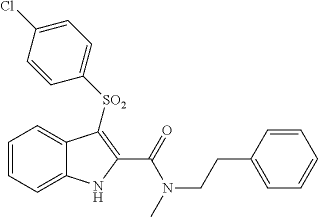 | ¹H-NMR (400 MHz, DMSO-d6) 55:45 rotamer ratio, δ = 12.81 (m, 1H), 8.04 (m, 2H), 7.84 (d, 1H, J = 8.1 Hz), 7.65 (m, 2H), 7.50 (d, 1H, J = 7.6 Hz), 7.10-7-40 (m, 6H), 3.74 (m, 1H), 3.34 (m, 1H), 3.09 (s, 1.35H), 2.96 (m, 2H), 2.85 (s, 1.65H). MS calcd. for C₂₄H₂₂ClN₂O₃S (M + H⁺) 453.1, found 453.2. |
| A26 | 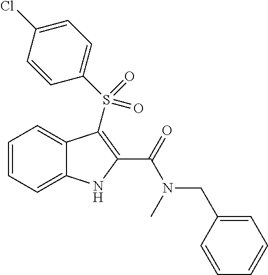 | ¹H-NMR (400 MHz, DMSO-d6) 68:32 rotamer ratio, δ = 12.95 (s, 0.32H), 12.89 (s, 0.68H), 8.06 (m, 2H), 7.85 (m, 1H), 7.66 (m, 1H), 7.23-7.53 (m, 8H), 4.80 (s, 1.36H), 4.42 (s, 0.64H), 2.94 (s, 0.96 H), 2.77 (s, 2.04H). MS calcd. for C₂₃H₂₀ClN₂O₃S (M + H⁺) 439.1, found 439.2. |
| A27 | 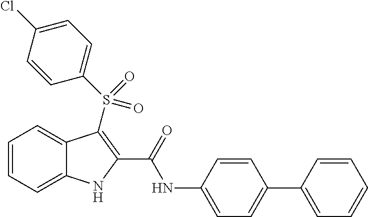 | ¹H-NMR (400 MHz, DMSO-d6) δ = 11.07 (s, 1H), 8.10 (m, 2H), 7.98 (d, 1H, J = 7.8 Hz), 7.85 (m, 2H), 7.75 (m, 2H), 7.69 (m, 4H), 7.55 (d, 1H, J = 7.9 Hz), 7.48 (m, 2H), 7.34 (m, 3H). MS calcd. for C₂₇H₂₀ClN₂O₃S (M + H⁺) 487.1, found 487.2. |
| A28 | 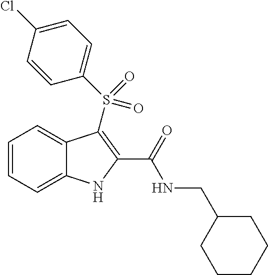 | ¹H-NMR (400 MHz, DMSO-d6) δ = 12.79 (s, 1H), 8.92 (t, 1H, J = 5.6 Hz), 8.03 (m, 2H), 7.94 (d, 1H, J = 7.8 Hz), 7.64 (m, 2H), 7.51 (m, 1H, J = 8.0 Hz), 7.29 (m, 2H), 3.19 (t, 2H, J = 6.3 Hz), 1.81 (m, 2H), 1.63 (m, 4H), 1.21 (m, 3H), 0.98 (m, 2H). MS calcd. for C₂₂H₂₄ClN₂O₃S (M + H⁺) 431.1, found 431.2. |
| A30 | 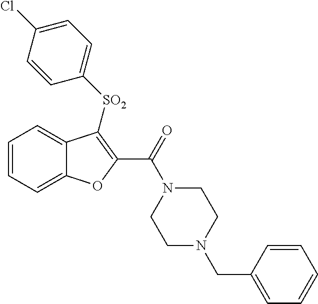 | ¹H-NMR (400 MHz, DMSO-d6) δ = 10.09 (bs, 1H), 8.13 (m, 2H), 7.92 (m, 1H), 7.77 (m, 3H), 7.40-7.61 (m, 7H), 2.70-4.70 (m, 10H). MS calcd. for C₂₆H₂₄ClN₂O₄S (M + H⁺) 495.1, found 495.2. |
| A31 | 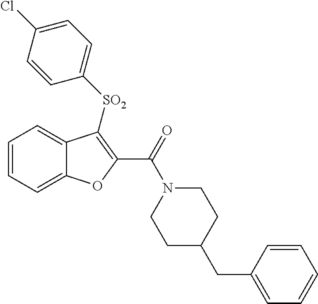 | ¹H-NMR (400 MHz, DMSO-d6) δ = 8.14 (m, 2H), 7.90 (m, 1H), 7.79 (d, 1H, J = 8.2 Hz), 7.75 (m, 2H), 7.54 (m, 1H), 7.49 (m, 1H), 7.28 (m, 2H), 7.18 (m, 3H), 4.47 (m, 1H), 3.41 (m, 1H), 3.13 (m, 1H), 2.88 (m, 1H), 2.55 (d, 2H, J = 7.2 Hz), 1.87 (m, 1H), 1.72 (m, 1H), 1.58 (m, 1H), 1.22 (m, 2H). MS calcd. for C₂₇H₂₅ClNO₄S (M + H⁺) 494.1, found 494.1. |
| A32 | 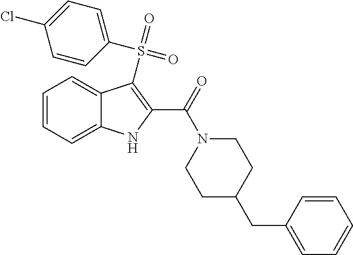 | ¹H-NMR (400 MHz, DMSO-d6) δ = 12.80 (s, 1H), 8.04 (d, J = 8.2 Hz, 2H), 7.82 (d, J = 7.6 Hz, 1H), 7.65 (m, 2H), 7.48 (d, J = 7.8 Hz, 1H), 7.27 (m, 4H), 7.18 (m, 3H), 4.55 (d, J = 12.9 Hz, 1H), 3.27 (d, J = 13.2 Hz, 1H), 3.04 (m, 1H), 2.81 (ddd, J = 12.8, 12.8, 2.6 Hz, 1H), 2.53 (d, J = 7.0 Hz, 2H), 1.84 (m, 1H), 1.71 (d, J = 12.2 Hz, 1H), 1.52 (d, J = 11.8 Hz, 1H), 1.28 (m, 2H). MS calcd. for C₂₇H₂₆N₂O₃S (M + H⁺) 493.1, found 493.2. |

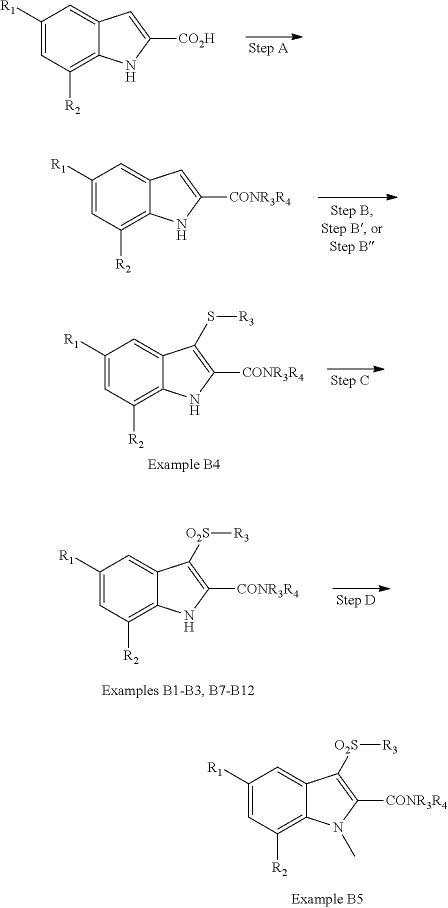

### Example B1

### (4-benzylpiperidin-1-yl)(3-(4-chlorophenylsulfonyl)-7-fluoro-1H-indol-2-yl)methanone

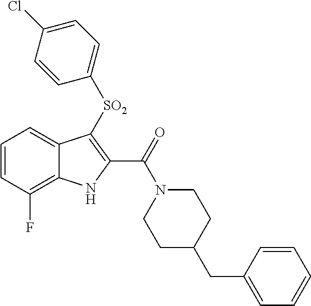

Step A. 7-Fluoro-1H-indole-2-carboxylic acid and 4 benzylpiperidine are reacted using HATU as the coupling reagent according to the procedure outlined in Example A1, Step D to afford (4-benzylpiperidin-1-yl)(7-fluoro-1H-indol-2-yl)methanone: MS calcd. for C₂₁H₂₂FN₂O (M+H⁺) 337.2, found 337.1.

Step B. (4-Benzylpiperidin-1-yl)(7-fluoro-1H-indol-2-yl)methanone is sulfenylated according to procedure in Example A1, Step A to afford (4-benzylpiperidin-1-yl)(3-(4-chlorophenylthio)-7-fluoro-1H-indol-2-yl)methanone: MS calcd. for C₂₇H₂₅ClFN₂OS (M+H⁺) 479.1, found 479.1.

Step C. Oxidation of (4-benzylpiperidin-1-yl)(3-(4-chlorophenylthio)-7-fluoro-1H-indol-2-yl)methanone is performed according to Example A1, Step B to afford (4-benzylpiperidin-1-yl)(3-(4-chlorophenylsulfonyl)-7-fluoro-1H-indol-2-yl)methanone B1: ¹H-NMR (600 MHz, DMSO-d6) δ=8.03 (m, 2H), 7.65 (m, 3H), 7.28 (m, 2H), 7.23 (m, 1H), 7.17 (m, 4H), 4.54 (m, 1H), 3.28 (m, 1H), 3.04 (m, 1H), 2.81 (m, 1H), 2.52 (m, 2H), 1.83 (m, 1H), 1.71 (m, 1H), 1.52 (m, 1H), 1.17-1-42 (m, 2H). MS calcd. for C₂₇H₂₅ClFN₂O₃S (M+H⁺) 511.1, found 511.1.

### Example B2

### (4-benzylpiperidin-1-yl)(3-benzylsulfonyl-1H-indol-2-yl)methanone

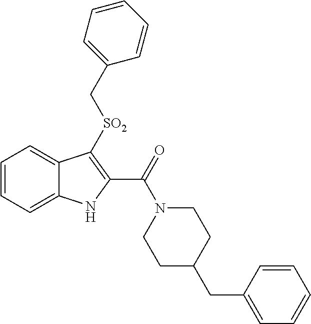

Step A. 1H-indole-2-carboxylic acid and 4-benzylpiperidine are reacted using HATU as the coupling reagent according to the procedure outlined in Example A1, Step D to afford (4-benzylpiperidin-1-yl)(1H-indol-2-yl)methanone: ¹H-NMR (400 MHz, DMSO-d6) δ=11.54 9 (s, 1H), 7.59 (m, 1H), 7.40 (m, 1H), 7.29 (m, 2H), 7.19 (m, 3H), 7.03 (m, 1H), 6.74 (m, 1H), 4.43 (m, 2H), 2.96 (m, 2H), 2.56 (m, 2H), 1.85 (m, 1H), 1.67 (m, 2H), 1.19 (m, 2H). MS calcd. for C₂₁H₂₃N₂O (M+H⁺) 319.2, found 319.4.

Step B′ (R₃=alkyl): A solution of NCS (32 mg, 0.32 mmol) in dry DCM (2 mL) is cooled to −78° C. BnSH (30 mg, 28 nL, 0.24 mmol) is added and the reaction mixture is warmed to 0° C. and stirred for 15 min. Then a solution of (4-benzylpiperidin-1-yl)(1H-indol-2-yl)methanone (64 mg, 0.2 mmol) is added. The reaction mixture is stirred at 0° C. for 1 h. H₂O (10 mL) is added and the mixture is extracted with DCM (3×10 mL), dried (Na₂SO₄), filtered and concentrated. The mixture is purified by flash chromatography on silica gel (hexanes/EtOAc gradient) to afford (4-benzylpiperidin-1-yl)(3-(benzylthio)-1H-indol-2-yl)methanone. MS calcd. for C₂₈H₂₉N₂OS (M+H⁺) 441.2, found 441.2.

Step C. Oxidation of (4-benzylpiperidin-1-yl)(3-(benzylthio)-1H-indol-2-yl)methanone is performed as described in Example A1, Step B to afford the title compound B2: ¹H-NMR (400 MHz, DMSO-d6) δ=12.65 (s, 1H), 7.44 (d, 1H, J=8.2 Hz), 7.39 (d, 1H, J=8.1 Hz), 7.28 (m, 2H), 7.19 (m 9H), 7.05 (t, 1H, J=7.4 Hz), 4.54 (s, 2H), 4.51 (m, 1H), 3.22 (m, 1H), 2.94 (m, 1H), 2.77 (m, 1H), 2.52 (m, 2H), 1.82 (m, 1H), 1.70 (m, 1H), 1.46 (m, 1H), 1.22 (m, 2H). MS calcd. for C₂₈H₂₉N₂O₃S (M+H⁺) 473.2, found 473.2.

### Example B3 and B4

### (4-benzylpiperidin-1-yl)(3-(cyclohexylthio)-1H-indol-2-yl)methanone and (4-benzylpiperidin-1-yl)(3-benzylsulfonyl-1H-indol-2-yl)methanone

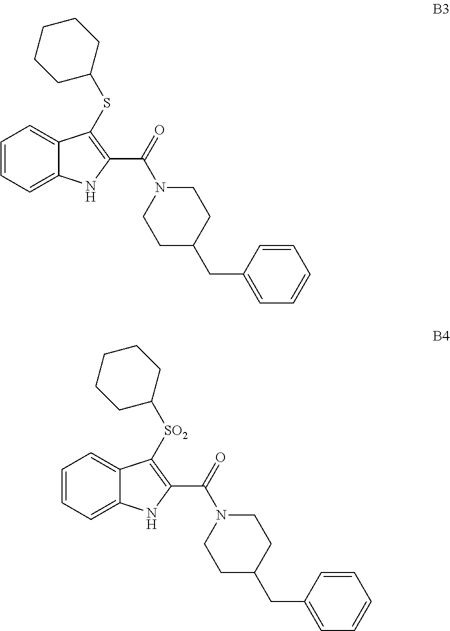

Step A: Step A is performed according to step A for example B1.

Step B″ (R₃=sterically crowded alkyl): A solution of (4-benzylpiperidin-1-yl)(1H-indol-2-yl)methanone (64 mg, 0.2 mmol) in DMA (0.5 mL) is degassed with nitrogen. Then N-(cyclohexylthio)phthalimide (98 mg, 0.32 mmol), MgBr₂ (18 mg, 0.1 mmol), and Et₃N (19.5 μL, 0.14 mmol) is added, and the reaction mixture is stirred under nitrogen for 19 h at 90° C. H₂O (10 mL) is added to the cold reaction mixture and the product is extracted with DCM (3×10 mL), dried (Na₂SO₄), and concentrated to yield the title compound B3 after purification by reverse phase HPLC (H₂O/MeCN gradient). ¹H-NMR (400 MHz, DMSO-d6) δ=11.83, (s, 1H), 7.85 (d, 1H, J=7.9 Hz), 7.37 (d, 1H, J=8.0 Hz), 7.28 (m, 2H), 7.18 (m, 4H), 7.12 (m, 1H), 4.51 (m, 1H), 3.50 (m, 1H), 3.01 (m, 1H), 2.78 (m, 2H), 2.53 (m, 2H), 1.43-1.84 (m, 8H), 1.17 (m, 7H). MS calcd. for C₂₇H₃₃N₂OS (M+H⁺) 433.2, found 433.2.

Step C: Step C is performed according to step A for example B1 to afford the title compound B4. ¹H-NMR (400 MHz, DMSO-d6) δ=12.68, (s, 1H), 7.79 (d, 1H, J=7.9 Hz), 7.49 (d, 1H, J=8.0 Hz), 7.27 (m, 4H), 7.18 (m, 3H), 4.47 (m, 1H), 3.21 (m, 1H), 3.02 (m, 2H), 2.73 (m, 1H), 2.52 (m, 2H), 1.03-2.03 (m, 15H). MS calcd. for C₂₇H₃₃N₂O₃S (M+H⁺) 465.2, found 465.2.

### Example B5

### (4-Benzylpiperidin-1-yl)(3-(4-chlorophenylsulfonyl)-1-methyl-1H-indol-2-yl)methanone

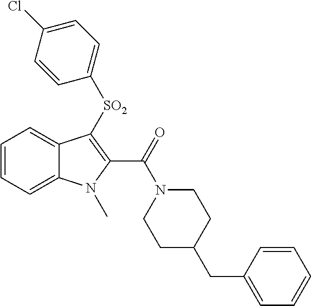

Step D. To a solution of (4-benzylpiperidin-1-yl)(1H-indol-2-yl)methanone (21 mg, 0.043 mmol) in THF (0.5 mL) is added 60% NaH in oil (8 mg 0.2 mmol) under a N₂ atmosphere and the mixture is stirred for 15 min at rt. MeI (28 mg, 12.5 μL, 0.2 mmol) is added and the reaction mixture is stirred for an additional 2.5 h at rt. H₂O (10 mL) is added and the mixture is extracted with DCM (3×10 mL), dried (Na₂SO₄), and concentrated. The title compound B5 is isolated after purification by reverse phase HPLC (H₂O/MeCN gradient). ¹H-NMR (400 MHz, DMSO-d6), rotamers of 54:46 ratio, δ=8.04 (m, 1.08H), 7.96 (m, 0.92H), 7.85 (d, 0.54H, J=8.0 Hz), 7.83 (d, 0.46 H, J=8.0 Hz), 7.65 (m, 3H), 7.37 (m, 1H), 7.29 (m, 3H), 7.18 (m, 3H), 4.58 (m, 1H), 3.75 (s, 1.38H), 3.68 (s, 1.62H), 3.27 (m, 1H), 3.14 (m, 1.08H), 3.04 (m, 0.92H), 2.87 (m, 1H), 2.57 (dd, 0.92H, J=3.1, 7.2 Hz), 2.53 (m, 1.08H), 1.84 (m, 1H), 1.12-1.78 (m, 4H). MS calcd. for C₂₈H₂₈ClN₂O₃S (M+H⁺) 507.1, found 507.1.

By repeating the procedure described in the above examples B1-B5, using appropriate starting materials, the following compounds of Formula I, as identified in Table 2, are obtained.

TABLE 2

| | | Physical Data |
|---|---|---|
| Compound | Compound | ¹H NMR 400 MHz (DMSO-d₆) |
| Number | Structure | and/or MS (m/z) |
| B7 | 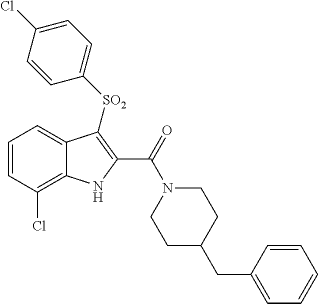 | ¹H-NMR (600 MHz, DMSO-d6) 53:47 rotamer ratio, δ = 8.06 (d, 1.06H, J = 8.5 Hz), 7.99 (d, 0.94 H, J = 8.4 Hz), 7.94 (m, 1H), 7.66 (m, 2H), 7.40 (m, 1H), 7.27 (m, 3H), 7.18 (m, 3H), 4.55 (m, 1H), 3.27 (m, 1H), 3.03 (m, 1H), 2.81 (m, 1H), 2.53 (m, 2H), 1.85 (m, 1H), 1.70 (m, 1H), 1.52 (m, 1H), 1.22-1.40 (m, 2H). MS calcd. for C₂₇H₂₅Cl₂N₂O₃S (M + H⁺) 527.1, found 527.0. |
| B8 | 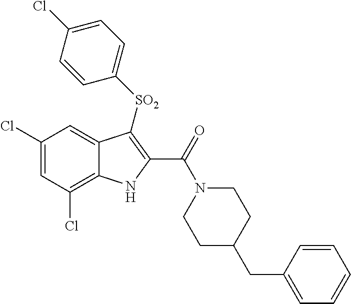 | ¹H-NMR (600 MHz, DMSO-d6) 53:47 rotamer ratio, δ = 8.08 (d, 1.06H, J = 8.3 Hz), 8.01 (d, 0.94 H, J = 8.3 Hz), 7.78 (m, 1H), 7.68 (m, 2H), 7.55 (m, 1H), 7.28 (m, 2H), 7.18 (m, 3H), 4.53 (m, 1H), 3.24 (m, 1H), 2.97 (m, 1H), 2.81 (m, 1H), 2.53 (m, 2H), 1.84 (m, 1H), 1.70 (m, 1H), 1.51 (m, 1H), 1.33 (m, 1H), 1.24 (m 1H). MS calcd. for C₂₇H₂₄Cl₃N₂O₃S (M + H⁺) 561.1, found 561.1. |
| B9 | 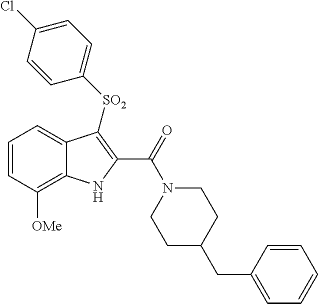 | ¹H-NMR (600 MHz, DMSO-d6) rotamers, δ = 12.91 and 12.81 (s, 1H), 8.01 (m, 2H), 7.64 (m, 2H), 7.37 (m, 1H), 7.28 (m, 2H), 7.17 (m, 4H), 6.85 (d, 1H, J = 7.7 Hz), 4.53 (m, 1H), 3.92 and 3.90 (s, 3H), 3.23 (m, 1H), 3.00 (m, 1H), 2.77 (m, 1H), 2.53 (m, 2H), 1.83 (m, 1H), 1.68 (m, 1H), 1.49 (m, 1H), 1.34 (m, 1H), 1.24 (m, 1H). MS calcd. for C₂₈H₂₈ClN₂O₄S (M + H⁺) 523.1, found 523.2. |
| B10 | 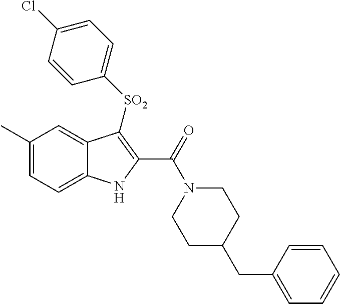 | ¹H-NMR (600 MHz, DMSO-d6) δ = 12.66 (s, 1H), 8.03 (d, 1H, J = 7.6 Hz), 7.65 (m, 2H), 7.60 (s, 1H), 7.36 (d, 1H, J = 8.3 Hz), 7.28 (m, 2H), 7.18 (m, 3H), 7.11 (dd, 1H, J = 1.4, 8.4 Hz), 4.53 (m, 1H), 3.25 (m, 1H), 3.02 (m, 1H), 2.79 (m, 1H), 2.53 (m, 2H), 2.40 (s, 3H), 1.84 (m, 1H), 1.70 (m, 1H), 1.51 (m, 1H), 1.16-1.39 (m, 2H). MS calcd. for C₂₈H₂₈ClN₂O₃S (M + H⁺) 507.1, found 507.2. |
| B11 | 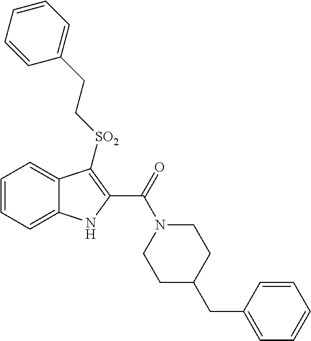 | ¹H-NMR (400 MHz, DMSO-d6) δ = 12.73 (s, 1H), 7.89 (d, 1H, J = 7.8 Hz), 7.51 (d, 1H, J = 8.0 Hz), 7.13-7.35 (m, 12H), 4.49 (m, 1H), 3.55 (m, 2H), 3.29 (m, 1H), 3.01 (m, 1H), 2.91 (m, 2H), 2.76 (m, 1H), 2.52 (m, 2H), 1.82 (m, 1H), 1.68 (m, 1H), 1.50 (m, 1H), 1.30 (m, 1H), 1.19 (m, 1H). MS calcd. for C₂₉H₃₁N₂O₃S (M + H⁺) 487.2, found 487.2. |
| B12 | 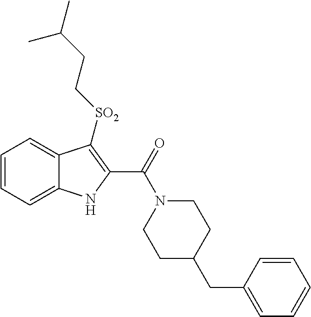 | 1H-NMR (400 MHz, DMSO-d6) δ = 12.66 (s, 1H), 7.83 (d, 1H, J = 7.8 Hz), 7.50 (d, 1H, J = 8.0 Hz), 7.28 (m, 4H), 7.18 (m, 3H) 4.48 (m, 1H), 3.24 (m, 3H), 3.00 (m, 1H), 2.74 (m, 1H), 2.52 (m, 2H), 1.82 (m, 1H), 1.67 (m, 1H), 1.52 (m, 4H), 1.29 (m, 1H), 1.17 (m, 1H). MS calcd. for C₂₆H₃₃N₂O₃S (M + H+) 453.2, found 453.2. |

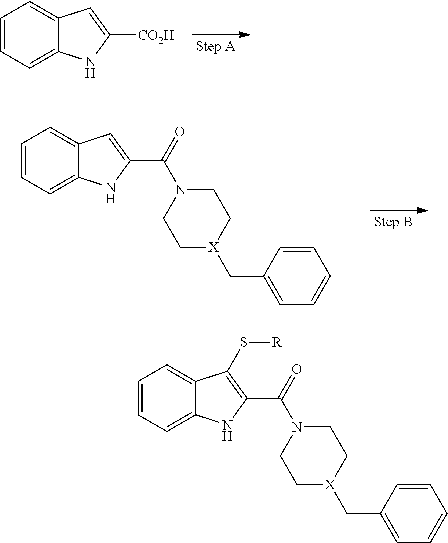

### Example C1

### (4-Benzylpiperidin-1-yl)(3-(phenylthio)-1H-indol-2-yl)methanone

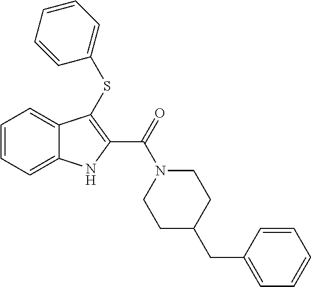

Step A (X═CH or N): A solution of indole-2-carboxylic acid (994 mg, 6.17 mmol) in THF (15 mL) is treated with CDI (1.50 g, 9.25 mmol) and stirred for 1 h at rt. 4-benzylpiperidine (3.25 mL, 0.0185 mol) is introduced and the reaction is continued to stir overnight. The reaction is poured into H₂O, extracted with EtOAc and washed with sat. aqu. NaHCO₃. The organic phase is separated, dried (MgSO₄), filtered, and concentrated. The crude residue is purified via flash chromatography (SiO₂) using a linear solvent gradient of 0-100% EtOAc in hexane to afford the desired (4-benzylpiperidin-1-yl)(1H)-indol-2-yl)methanone. ¹H-NMR (400 MHz, CDCl₃) δ=9.94 (s, 1H), 7.65 (d, J=8.0 Hz, 1H), 7.45 (d, J=8.3 Hz, 1H), 7.31 (m, 2H), 7.25 (m, 2H), 7.15 (m, 3H), 6.77 (d, J=1.5 Hz, 1H), 4.75 (d, J=13.2 Hz, 2H), 2.99 (m, 1H), 2.61 (d, J=7.1 Hz, 2H), 1.89 (m, 1H), 1.81 (m, 3H), 1.34 (dddd, J=12.7, 12.7, 12.7, 4.1 Hz, 2H); MS calcd. for C₂₁H₂₃N₂O (M+H⁺) 319.4, found 319.6.

Step B (X═CH or N): A solution of (4-benzylpiperidin-1-yl)(1H)-indol-2-yl)methanone (67 mg, 0.21 mmol) in DMF (0.5 mL) is treated with cesium carbonate (89 mg, 0.25 mmol) followed by phenyl disulfide (69 mg, 0.31 mmol) and heated to 100° C. The reaction mixture is maintained at this temperature for 2.5 h, then cooled to rt and poured into H₂O. The reaction is extracted with EtOAc. The organic layer is separated, dried (MgSO₄), filtered, and concentrated. The crude residue is purified via column chromatography (SiO₂) using a linear solvent elution system of 0-65% EtOAc in hexanes to afford the desired (4-benzylpiperidin-1-yl)(3-(phenylthio)-1H-indol-2-yl)methanone C1. ¹H-NMR (400 MHz, DMSO-d6) δ=12.13 (s, 1H), 7.47 (d, J=8.2 Hz, 1H), 7.38 (d, J=7.9 Hz, 1H), 7.22 (m, 6H), 7.10 (m, 4H), 7.04 (m, 2H), 4.46 (d, J=11.6 Hz, 1H), 3.48 (m, 1H), 2.93 (m, 1H), 2.73 (m, 1H), 2.45 (m, 2H), 1.75 (m, 1H), 1.69 (m, 1H), 1.32 (m, 1H), 1.07 (m, 2H); MS calcd. for C₂₇H₂₇N₂OS (M+H⁺) 427.6, found 427.2.

By repeating the procedure described in the above examples C1, using appropriate starting materials, the following compounds of Formula I, as identified in Table 3, are obtained.

TABLE 3

| | | Physical Data |
|---|---|---|
| Compound | Compound | ¹H NMR 400 MHz (DMSO-d₆) |
| Number | Structure | and/or MS (m/z) |
| C2 | 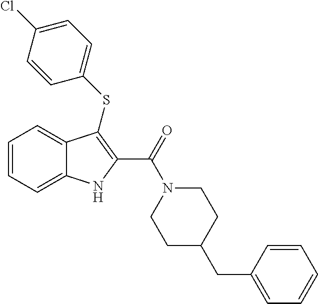 | ¹H-NMR (400 MHz, DMSO-d6) δ = 12.19 (s, 1H), 7.47 (d, J = 8.2 Hz, 1H), 7.37 (d, J = 8.1 Hz, 1H), 7.26 (m, 5H), 7.15 (m, 1H), 7.10 (m, 1H), 7.04 (m, 2H), 4.46 (d, J = 11.0 Hz, 1H), 3.45 (d, J = 11.4 Hz, 1H), 2.92 (m, 1H), 2.73 (m, 1H), 2.50 (m, 1H), 2.40 (m, 1H), 1.71 (m, 2H), 1.30 (m, 1H), 1.09 (m, 1H), 0.95 (m, 1H); MS calcd. for C₂₇H₂₅ClN₂OS (M⁺) 461.0, found 461.2.0. |
| C3 | 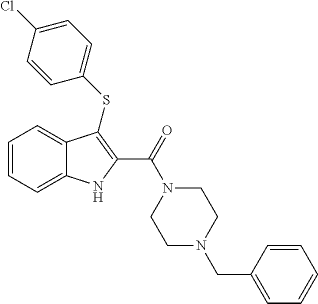 | MS calcd. for C₂₆H₂₄ClN₃OS (M⁺) 462.0, found 462.2. |

### Biological Assays

Generation of Stable Cell Line

Flp-In-CHO cells (Invitrogen, Cat. #R758-07) are maintained in Ham's F12 medium supplemented with 10% fetal bovine serum, 1% antibiotic mixture and 2 mM L-glutamine. The cells are transfected with a DNA mixture containing human GPR119 in pcDNA5/FRT vector and the pOG44 vector (1:9) using Fugene6 (Roche), according to the manufacturer's instruction. After 48 hours, the medium is changed to medium supplemented with 400 μg/ml hygromycin B to initiate the selection of stably transfected cells.

Cyclic AMP Assay in Stable Cell Line

To test the activity of compounds of the invention, Flp-In-CHO-hGPR119 cells are harvested and resuspended in DMEM plus 3% lipid-depleted fetal bovine serum. Forth μl of cells are plated in 384 well plates at a density of 15,000 cells/well. IBMX (3-isobutyl-1-methyl-xanthine) is added to the cells to a final concentration of 1 mM, followed by the addition of 500 nl of the compound to be tested. The cells are incubated at 37° C. for 30 minutes. Equal volume (20 μl) of the HTRF reagents, anti-cAMP-Cryptate and cAMP-XL665, are added to the cells. The plates are incubated at room temperature for 1 hour and read on a HTRF reader according to the manufacturer's instruction.

Compounds of Formula I, in free form or in pharmaceutically acceptable salt form, produced a concentration-dependent increase in intracellular cAMP level. Compound of the invention show an EC₅₀ of between 1×10⁻⁵ and 1×10⁻¹⁰M, preferably less than 500 nM, more preferably less than 100 nM. Specific EC₅₀ data is presented for some of the compounds of the invention in the table, infra.

Table of Biological Activity

| Example | CHO-GPR119-HTRF (3158) μM |
|---|---|
| A1 | 0.012 |
| A2 | 0.007 |
| A3 | 0.009 |
| A4 | 0.010 |
| A5 | 0.017 |
| A6 | 0.018 |
| A7 | 0.018 |
| A11 | 0.032 |
| A12 | 0.040 |
| A19 | 0.533 |
| A20 | 0.192 |
| A22 | 0.435 |
| A23 | 0.396 |
| A24 | 0.574 |
| A29 | 0.054 |
| A30 | 0.187 |
| B1 | 0.022 |
| B2 | 0.21 |
| B3 | 0.755 |
| B4 | 0.149 |
| B5 | 0.169 |
| B11 | 0.042 |
| C1 | 0.454 |
| C2 | 0.180 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 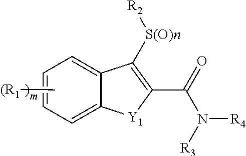
in which:
m is selected from 0, 1, 2, 3 and 4;
n is selected from 0, 1 and 2;
R₁ is selected from halo, halo-substituted-C₁₋₆alkyl, C₁₋₆alkoxy, halo-substituted-C₁₋₆alkoxy and C₁₋₆alkyl;
R₂ is selected from C₆₋₁₀aryl-C₀₋₄alkyl, C₅₋₁₀heteroaryl-C₀₋₄alkyl, C₃₋₁₂cycloalkyl-C₀₋₄alkyl, C₃₋₈heterocycloalkyl-C₀₋₄alkyl and C₁₋₆alkyl; wherein any aryl, heteroaryl, cycloalkyl, heterocycloalkyl or alkyl of R₂ can be optionally substituted with 1 to 3 radicals independently selected from halo, halo-substituted-C₁₋₆alkyl, C₁₋₆alkoxy, halo-substituted-C₁₋₆alkoxy, C₁₋₆alkyl and C₆₋₁₀aryl;
R₃ is selected from hydrogen and C₁₋₆alkyl;
R₄ is selected from —X₁R₅ and —X₁OR₅; wherein X₁ is selected from a bond, —C(O)—, —NR₆— and C₁₋₆alkylene; R₅ is selected from C₆₋₁₀aryl, C₁₋₁₀heteroaryl, C₃₋₈heterocycloalkyl and C₃₋₁₂cycloalkyl; R₆ is selected from hydrogen and C₁₋₆alkyl;
or R₃ and R₄ together with the nitrogen atom to which R₃ and R₄ are attached form C₃₋₈heterocycloalkyl; wherein said aryl or cycloalkyl of R₄ or said heterocycloalkyl of the combination of R₃ and R₄ can be optionally substituted with 1 to 3 radicals independently selected from —X₂R₇, —X₂C(O)R₇, —X₂S(O)₀₋₂R₇—X₂NR₈X₃R₇ and —X₂OR₇; wherein X₂ and X₃ are independently selected from a bond and C₁₋₄alkylene; R₇ is selected from C₆₋₁₀aryl, C₃₋₁₂cycloalkyl, C₁₋₁₀heteroaryl and C₃₋₈heterocycloalkyl; wherein said aryl, heteroaryl, cycloalkyl and heterocycloalkyl of R₇ is optionally substituted with 1 to 3 radicals independently selected from halo, hydroxy, nitro, cyano, halo-substituted-C₁₋₆alkyl, C₁₋₆alkoxy, halo-substituted-C₁₋₆alkoxy, C₁₋₆alkyl, C₆₋₁₀aryl and —C(O)R₉; wherein R₈ is selected from hydrogen and C₁₋₆alkyl; R₉ is selected from hydrogen and C₁₋₆alkyl;
Y₁ is selected from O and NR₁₀; wherein R₁₀ is selected from hydrogen and C₁₋₆alkyl; or the pharmaceutically acceptable salts thereof.

2. The compound of claim 1 in which:
m is selected from 0, 1 and 2;
n is selected from 0, 1 and 2;
R₁ is selected from halo, halo-substituted-C₁₋₆alkyl, C₁₋₆alkoxy, halo-substituted-C₁₋₆alkoxy and C₁₋₆alkyl;
R₂ is selected from C₆₋₁₀aryl-C₀₋₄alkyl, C₃₋₁₂cycloalkyl-C₀₋₄alkyl, and C₁₋₆alkyl; wherein any aryl, cycloalkyl or alkyl of R₂ can be optionally substituted with 1 to 3 radicals independently selected from halo, halo-substituted-C₁₋₆alkyl, C₁₋₆alkoxy, halo-substituted-C₁₋₆alkoxy and C₁₋₆alkyl;
R₃ is selected from hydrogen and C₁₋₆alkyl;
R₄ is selected from —X₁R₅ and —X₁OR₅; wherein X₁ is selected from a bond, —C(O)—, —NR₆— and C₁₋₆alkylene; R₅ is selected from C₆₋₁₀aryl, C₁₋₁₀heteroaryl and C₃₋₁₂cycloalkyl; R₆ is selected from hydrogen and C₁₋₆alkyl;
or R₃ and R₄ together with the nitrogen atom to which R₃ and R₄ are attached form C₃₋₈heterocycloalkyl; wherein said aryl or cycloalkyl of R₄ or said heterocycloalkyl of the combination of R₃ and R₄ can be optionally substituted with 1 to 3 radicals independently selected from —X₂R₇, —X₂C(O)R₇, —X₂S(O)₀₋₂R₇—X₂NR₈X₃R₇ and —X₂OR₇; wherein X₂ and X₃ are independently selected from a bond and C₁₋₄alkylene; R₇ is selected from C₆₋₁₀aryl, C₃₋₁₂cycloalkyl, C₁₋₁₀heteroaryl and C₃₋₈heterocycloalkyl; wherein said aryl, heteroaryl, cycloalkyl and heterocycloalkyl of R₇ is optionally substituted with 1 to 3 radicals independently selected from halo, hydroxy, nitro, cyano, halo-substituted-C₁₋₆alkyl, C₁₋₆alkoxy, halo-substituted-C₁₋₆alkoxy, C₁₋₆alkyl, C₆₋₁₀aryl and —C(O)R₉; wherein R₈ is selected from hydrogen and C₁₋₆alkyl; R₉ is selected from hydrogen and C₁₋₆alkyl; and
Y₁ is selected from O and NR₁₀; wherein R₁₀ is selected from hydrogen and C₁₋₆alkyl.

3. The compound of claim 2 in which: R₁ is selected fluoro, chloro, bromo, methyl, trifluoromethyl and methoxy; and R₂ is selected from phenyl, benzyl, cyclohexyl, phenethyl and isopentyl; wherein said phenyl, benzyl, cyclohexyl and phenethyl is optionally substituted with a halo radical.

4. The compound of claim 3 in which: R₃ is selected from hydrogen and methyl; and R₄ is selected from phenoxy-ethyl, benzyl, phenethyl, phenyl-butyl, biphenyl and cyclohexyl-methyl; or R₃ and R₄ together with the nitrogen atom to which R₃ and R₄ are attached form piperidinyl or piperazinyl; wherein said piperidinyl or piperazinyl are optionally substituted with a group selected from benzyl, phenoxy, phenyl-thio, phenethyl, cyclohexyl-methyl, benzo[d][1,3]dioxolyl-methyl, phenyl-carbonyl, benzyl-(methyl)-amino and pyridinyl-methyl; wherein said benzyl, phenoxy, phenyl-thio, phenethyl, cyclohexyl-methyl, benzo[d][1,3]dioxolyl-methyl, phenyl-carbonyl, benzyl-(methyl)-amino or pyridinyl-methyl substituents of the combination of R₃ and R₄ are further optionally substituted by 1 to 3 radicals independently selected from chloro, fluoro, bromo, methyl, trifluoromethyl, t-butyl, methoxy and formyl.

5. The compound of claim 4 in which Y₁ is selected from O and NR₁₀; wherein R₁₀ is selected from hydrogen and methyl.

6. The compound of claim 5 selected from: (4-benzylpiperidin-1-yl)(3-(4-chlorophenylsulfonyl)-5-fluoro-1H-indol-2-yl)methanone; (3-(4-Chlorophenylsulfonyl)benzofuran-2-yl)(4-(4-(trifluoromethyl)phenoxy)piperidin-1-yl)methanone; (4-(2-chloro-6-fluorobenzyl)piperazin-1-yl)(3-(4-chlorophenylsulfonyl)-1H-indol-2-yl)methanone; (3-(4-chlorophenylsulfonyl)-1H-indol-2-yl)(4-(p-tolyloxy)piperidin-1-yl)methanone; (3-(4-chlorophenylsulfonyl)-1H-indol-2-yl)(4-(4-(trifluoromethyl)phenoxy)piperidin-1-yl)methanone; (4-(4-chlorobenzyl)piperazin-1-yl)(3-(4-chlorophenylsulfonyl)-1H-indol-2-yl)methanone; (3-(4-chlorophenylsulfonyl)-1H-indol-2-yl)(4-(p-tolylthio)piperidin-1-yl)methanone; (3-(4-chlorophenylsulfonyl)-1H-indol-2-yl)(4-phenethylpiperidin-1-yl)methanone; (4-(4-tert-butylbenzyl)piperazin-1-yl)(3-(4-chlorophenylsulfonyl)-1H-indol-2-yl)methanone; (3-(4-chlorophenylsulfonyl)-1H-indol-2-yl)(4-(4-methoxybenzyl)piperazin-1-yl)methanone; (4-benzylpiperazin-1-yl)(3-(4-chlorophenylsulfonyl)-1H-indol-2-yl)methanone; (3-(4-chlorophenylsulfonyl)-1H-indol-2-yl)(4-(cyclohexylmethyl)piperazin-1-yl)methanone; (4-(benzo[d][1,3]dioxol-5-ylmethyl)piperazin-1-yl)(3-(4-chlorophenylsulfonyl)-1H-indol-2-yl)methanone; (4-benzylpiperidin-1-yl)(5-chloro-3-(4-chlorophenylsulfonyl)-1H-indol-2-yl)methanone; (4-benzoylpiperidin-1-yl)(3-(4-chlorophenylsulfonyl)-1H-indol-2-yl)methanone; (4-benzylpiperidin-1-yl)(3-(4-chlorophenylsulfonyl)-5,7-difluoro-1H-indol-2-yl)methanone; 4-(1-(3-(4-chlorophenylsulfonyl)-1H-indole-2-carbonyl)piperidin-4-yloxy)benzaldehyde; (3-(4-chlorophenylsulfonyl)-1H-indol-2-yl)(4-phenethylpiperazin-1-yl)methanone; (4-benzylpiperidin-1-yl)(3-(4-chlorophenylsulfonyl)-7-(trifluoromethyl)-1H-indol-2-yl)methanone; (4-(benzyl(methyl)amino)piperidin-1-yl)(3-(4-chlorophenylsulfonyl)-1H-indol-2-yl)methanone; 3-(4-chlorophenylsulfonyl)-N-methyl-N-(2-phenoxyethyl)-1H-indole-2-carboxamide; (4-benzylpiperidin-1-yl)(5-bromo-3-(4-chlorophenylsulfonyl)-1H-indol-2-yl)methanone; (3-(4-chlorophenylsulfonyl)-1H-indol-2-yl)(4-(pyridin-4-ylmethyl)piperazin-1-yl)methanone; 3-(4-chlorophenylsulfonyl)-N-(4-phenylbutyl)-1H-indole-2-carboxamide; (3-(4-chlorophenylsulfonyl)-1H-indol-2-yl)(piperidin-1-yl)methanone; 3-(4-chlorophenylsulfonyl)-N-methyl-N-phenethyl-1H-indole-2-carboxamide; N-benzyl-3-(4-chlorophenylsulfonyl)-N-methyl-1H-indole-2-carboxamide; N-(biphenyl-4-yl)-3-(4-chlorophenylsulfonyl)-1H-indole-2-carboxamide; 3-(4-chlorophenylsulfonyl)-N-(cyclohexylmethyl)-1H-indole-2-carboxamide; (4-benzylpiperazin-1-yl)(3-(4-chlorophenylsulfonyl)benzofuran-2-yl)methanone; (4-benzylpiperidin-1-yl)(3-(4-chlorophenylsulfonyl)benzofuran-2-yl)methanone; (4-benzylpiperidin-1-yl)(3-(4-chlorophenylsulfonyl)-1H-indol-2-yl)methanone; (4-benzylpiperidin-1-yl)(3-(4-chlorophenylsulfonyl)-7-fluoro-1H-indol-2-yl)methanone; (4-benzylpiperidin-1-yl)(3-benzylsulfonyl-1H-indol-2-yl)methanone; (4-benzylpiperidin-1-yl)(3-(cyclohexylthio)-1H-indol-2-yl)methanone; (4-benzylpiperidin-1-yl)(3-benzylsulfonyl-1H-indol-2-yl)methanone; (4-Benzylpiperidin-1-yl)(3-(4-chlorophenylsulfonyl)-1-methyl-1H-indol-2-yl)methanone; (4-benzylpiperidin-1-yl)(7-chloro-3-(4-chlorophenylsulfonyl)-1H-indol-2-yl)methanone; (4-benzylpiperidin-1-yl)(5,7-dichloro-3-(4-chlorophenylsulfonyl)-1H-indol-2-yl)methanone; (4-benzylpiperidin-1-yl)(3-(4-chlorophenylsulfonyl)-7-methoxy-1H-indol-2-yl)methanone; (4-benzylpiperidin-1-yl)(3-(4-chlorophenylsulfonyl)-5-methyl-1H-indol-2-yl)methanone; (4-benzylpiperidin-1-yl)(3-(phenethylsulfonyl)-1H-indol-2-yl)methanone; (4-benzylpiperidin-1-yl)(3-(isopentylsulfonyl)-1H-indol-2-yl)methanone; (4-Benzylpiperidin-1-yl)(3-(phenylthio)-1H-indol-2-yl)methanone; (4-benzylpiperidin-1-yl)(3-(4-chlorophenylthio)-1H-indol-2-yl)methanone; and (4-benzylpiperazin-1-yl)(3-(4-chlorophenylthio)-1H-indol-2-yl)methanone.

7. A pharmaceutical composition comprising a therapeutically effective amount of a compound of claim 1 in combination with a pharmaceutically acceptable excipient.

8. A method for modulating GPR119 activity, comprising administering to a system or a subject in need thereof, a therapeutically effective amount of the compound of claim 1 or pharmaceutically acceptable salts or pharmaceutical compositions thereof, thereby modulating said GPR119 activity.

9. The method of claim 8, wherein the compound of claim 1 directly contacts GPR119.

10. The method of claim 11, wherein the contacting occurs in vitro or in vivo.

11. A method for treating a disease or condition wherein modulation of GPR119 activity can prevent, inhibit or ameliorate the pathology and/or symptomology of the disease or condition, comprising administering to a subject a therapeutically effective amount of the compound of claim 1 or pharmaceutically acceptable salts or pharmaceutical compositions thereof.

12. The method of claim 11, wherein said disease or condition is selected from obesity, type 1 diabetes, type 2 diabetes mellitus, hyperlipidemia, idiopathic type 1 diabetes, latent autoimmune diabetes in adults, early-onset type 2 diabetes, youth-onset atypical diabetes, maturity onset diabetes of the young, malnutrition-related diabetes and gestational diabetes.

13. The method of claim 11, wherein said disease or condition is selected from coronary heart disease, ischemic stroke, restenosis after angioplasty, peripheral vascular disease, intermittent claudication, myocardial infarction, dyslipidemia, post-prandial lipemia, conditions of impaired glucose tolerance, conditions of impaired fasting plasma glucose, metabolic acidosis, ketosis, arthritis, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance.
